(19) United States
(12) Patent Application Publication
(10) Pub. No.: US 2014/0024815 A1
(43) Pub. Date: Jan. 23, 2014

(54) DENDRIMERS WITH INTERIOR AND EXTERIOR FUNCTIONALITIES COMPRISING OF AZIDE OR ALKYNE GROUPS FOR POST-FUNCTIONALIZATION BY HUISGEN CLICK CYCLOADDITION

(71) Applicant: POLYMER FACTORY SWEDEN AB, Nacka (SE)

(72) Inventors: Anders HULT, Taby (SE); Michael MALKOCH, Taby (SE); Per ANTONI, Taby (SE)

(73) Assignee: POLYMER FACTORY SWEDEN AB, Nacka (SE)

(21) Appl. No.: 14/036,520

(22) Filed: Sep. 25, 2013

Related U.S. Application Data
(62) Division of application No. 12/990,676, filed on Nov. 17, 2010.
(60) Provisional application No. 61/051,212, filed on May 7, 2008.

(30) Foreign Application Priority Data
May 7, 2008 (SE) 0801015-9

(51) Int. Cl. C07D 407/14; C07C 245/04; C07D 405/14; C07D 405/12; C07C 229/30; C07C 255/20

(52) U.S. Cl. CPC C07D 407/14; C07C 229/30; C07C 255/20; C07D 405/14; C07D 405/12; C07C 245/04
USPC 534/796; 549/370; 560/169; 558/442; 548/255

(57) ABSTRACT

A dendritic structure includes a core and repeating units, wherein the repeating units comprise units of the type ABₓC_{y}, wherein x is 2, 3, or 4, wherein y is 1, 2, or 3, wherein C is selected from the group consisting of azides and alkynes, and wherein every repeating unit is bound to at least one other unit with at least one bond selected from the group consisting of the group consisting of an ester, an amide, a thioether, an ether, a urethane, an amine, and an imine. Advantages include that a “one-pot” growth of the dendritic structure is possible. It is possible to have more functional groups in a dendritic structure. It is possible to add different functional groups simultaneously both to an inner layer and to an outer layer in a dendritic structure.

### TECHNICAL FIELD

The present invention relates to dendritic structures, which can be functionalized both in the interior and in the exterior.

### BACKGROUND

Dendritic structures have gained attention since their dawn in 1980's. Dendrimers are elegant, fractal-like, structures and have been investigated since they may act as scaffolds that are easily post functionalized to fit various needs and can be used as 3D-objects in nanotechnology. Their character is desired in cutting edge biological technologies, including for instance MRI agents and drug delivery carriers. Dendritic structures are used within many different fields and applications.

Traditionally dendritic structures are manufactured by reacting monomers of ABₓ-type. A typical example is a monomer of AB₂-type monomer where A is an acid functionality and B is a hydroxyl functionality. The result of the reaction is a branched tree-like polymer structure, referred to as a dendritic polymer structure.

SE 468 771 to Perstorp AB discloses a dendritic macromolecule.

Dendrimers and dendritic polymer structures comprising functional groups are well known. Dendrimers with different functional groups in different layers are also known, see for instance W. R. Dichtel, S. Hecht, J. M. J. Fréchet: “Functionally Layered Dendrimers: A New Building Block and its Application to the Synthesis of Multichromophoric Light-Harvesting Systems” Org. Lett. 2005, 7, 4451-4454.

Dendrimers with dual functionalization of the outermost layer are also known. Goodwin, A. P., Lam, S. S., and Fréchet, J. M. J.: “Rapid, Efficient Synthesis of Hetherobifunctional Biodegradable Dendrimers” J. Am. Chem. Soc. 2007, 129, 22, 6994-6995.

WO 2006/005046 discloses the use of click chemistry, which in this case involves ligation of terminal acetylenes and azides, for the synthesis of triazole dendrimers.

WO 2007/012001 discloses a method for making di-block dendrimers using click chemistry.

U.S. Pat. No. 6,376,637 discloses a process for making dendritic polyurethanes by reacting diisocyanates with compounds containing at least two groups which are reactive toward isocyanates, typically hydroxyl groups, at least one of the reactants contains functional groups having a different reactivity compared to the other reactant and the reaction conditions are selected so that only certain reactive groups react in each reaction step.

WO 02/077037 discloses dendritic polymers comprising specific interior and exterior groups.

C. O. Liang and J. M. J. Fréchet in Macromolcules 2005, 38, 6276-6284 disclose post functionalization in the inner part of a dendrimer. There is a mentioned group which may be functionalized after manufacture of the dendrimers. There is shown a dendrimer comprising internal allyl groups. The method involves use of ruthenium catalysis.

A. V. Ambade and A. Kumar in J. Polym. Sci., Part A, Polymer Chemistry, 2004, 42, 5134-5145 disclose synthesis of functionalizable hyperbranched structures comprising azides. It is disclosed that the azide groups can be used to bind groups such as drug precursors etc.

G. R. Newkome, G. R. Baker, C. N. Moorefield, B. D. Woosley, J. M. Shade in Polym. Prepr. (Am. Chem. Soc., Div. Polym. Chem.), August 1996, 415-416 disclose that dendrimers can be manipulated after manufacture.

X. Feng, D. Taton, R. Borsali, E. L. Chaikof, Y. Gnanou in J. Am. Chem. Soc., 2006, 128, 11551-11562 disclose dendrimer-like polymers with external hydroxyl functions and internal vinylic groups. There is disclosed dendrimers manufactured from monomers of the type AB₂C.

S. Hecht in J. Polym. Sci. Part A: Polym. Chem., 2003, 41, 1047-1058 discloses dendrimers manufactured from monomers of the type AB₂C where C is a functional group (FG) or a group which can be functionalized after manufacture of the dendrimers.

M. Malkoch, K. Schleicher, E. Drockenmuller, C. J. Hawker, T. P. Russell, P. Wu and v. v. Fokin in Macromolecules vol. 38, No. 9, 3663-3678 disclose functionalization of dendrimers using click chemistry. There is disclosed functionalization of for instance an acetyelene terminated dendrimer. There is not disclosed or suggested functionalization with different functional groups in the interior and in the exterior.

Future cutting edge solutions demand materials with more sophisticated properties and thus there is a need for more easily tailored dendrimers and improved methods for the manufacture of dendrimers.

Problems in the state of the art regarding post functionalizable dendritic polymer structures include how to improve the manufacturing process and how to simplify the functionalization as well as post functionalization. One problem in the state of the art for dendrimers manufactured using ruthenium catalysis and which can be functionalised in the interior after manufacture is that the chemistry is not as robust and tolerant as desired.

One problem in the state of the art is to how to provide a dendrimer which can be post functionalized with different functional groups both in at least one inner layer and in an outer layer.

### SUMMARY

It is an object of the present invention to obviate at least some of the disadvantages in the prior art and provide improved dendritic polymer structures as well as processes for their manufacture and use of the dendritic polymer structures.

In a first aspect there is provided a dendritic structure, said dendritic structure comprising a core and repeating units, wherein the repeating units comprise units of the type ABₓC_{y}, wherein x is 2, 3, or 4,

wherein y is 1, 2, or 3,
wherein C is selected from the group consisting of azides and alkynes, and
wherein every repeating unit is bound to at least one other unit with at least one bond selected from the group consisting of the group consisting of an ester, an amide, a thioether, an ether, a urethane, an amine, and an imine.

In a second aspect there is provided a method of manufacturing a dendritic structure comprising the steps

a) reacting at least two monomers of the type ABₓC_{y} with a core molecule, and
b) reacting the result from step a) with monomers of the type ABₓC_{y} to obtain a larger dendritic structure,
wherein x is 2, 3, or 4,
wherein y is 1, 2, or 3, and
wherein C is selected from the group consisting of azides and alkynes

In a third aspect there is provided a method of adding functional groups to a dendritic structure, wherein a functional group is added to a group C in the dendritic structure. The group C is selected from the group consisting of azides and alkynes.

In a fourth aspect there is provided a dendritic structure further comprising at least one functional group, characterized in that said at least one functional group is attached to an azide or alkyne in the dendritic structure.

In a fifth aspect there is provided use of a dendritic structure comprising functional groups.

In a sixth aspect there is provided a method for the manufacture of a particle comprising a crosslinking reaction of the dendritic structure according to the invention.

In a seventh aspect there is provided a particle manufactured from the dendritic structure according to the invention.

In an eight aspect there is provided a hydrogel manufactured from the dendritic structure according to the invention.

Further aspects and embodiments are defined in the appended claims, which are specifically incorporated herein by reference.

One advantage of an embodiment is that it is possible to use a “one-pot” growth of the dendritic structure.

One advantage is that there is provided the possibility to have more functional groups in a dendritic structure compared to prior art. When comparing the number of functional groups of a dendrimer based on ABₓC_{y}-monomers with a traditional dendrimer based ABₓ-monomers, it is evident that the intrinsic functionality provides a larger number of available functional groups for post-modification. For instance, a multifunctional dendrimer of the 5^{th} generation with a three functional core holds a total of 189 functional groups compared to its traditional analogue hold only 96 functional groups.

Another advantage is that there is provided the possibility of a synthesis which is very robust, can be performed in various solvents, performed at both ambient and elevated temperatures, performed at atmospheric pressure as well as elevated, performed in a variety of gases including oxygen, nitrogen, argon etc. In an embodiment the synthesis has a high yield making the manufacture economical.

A further advantage is that there is the possibility to exclude an activation step prior to post-functionalisation of a dendritic structure.

One advantage is that there is provided the possibility to add different types of functional groups simultaneously both to at least one inner layer and to the outer layer.

### DEFINITIONS

Before the invention is disclosed and described in detail, it is to be understood that this invention is not limited to particular monomers, compounds, configurations, method steps, substrates, and materials disclosed herein as such monomers, compounds, configurations, method steps, substrates, and materials may vary somewhat. It is also to be understood that the terminology employed herein is used for the purpose of describing particular embodiments only and is not intended to be limiting since the scope of the present invention is limited only by the appended claims and equivalents thereof.

It must be noted that, as used in this specification and the appended claims, the singular forms “a”, “an” and “the” include plural referents unless the context clearly dictates otherwise.

If nothing else is defined, any terms and scientific terminology used herein are intended to have the meanings commonly understood by those of skill in the art to which this invention pertains.

The term “about” as used in connection with a numerical value throughout the description and the claims denotes an interval of accuracy, familiar and acceptable to a person skilled in the art. Said interval is ±10%.

“Bond” is used herein to denote the phenomenon of atoms being held together in molecules by attraction of atoms.

“Crosslinking” is used herein to denote bonds that link one polymer chain to another. In this respect it is understood that a dendritic structure comprises polymer chains.

“Dendrimer” is used herein to denote repeatedly branched molecules and molecules. Dendrimers are monodisperse.

“Dendritic structure” is used herein to denote a branched structure. Examples of dendritic structures include but are not limited to dendrons, dendrimers, hyperbranched and dendronized polymers.

“Functionalization” is used herein to denote the addition of a functional group.

“Functional group” is used herein to denote specific groups of atoms within molecules that are responsible for characteristic chemical reactions and properties of the molecule.

“Hydrogel” is used herein to denote a network of polymer chains that are water-insoluble, in which water is the dispersion medium. In this respect it is understood that a dendritic structure comprises polymer chains.

“Monomer” is used herein to denote a molecule that may undergo a polymerisation reaction to become chemically bonded to other monomers to form a polymer.

“Polymer structure” is used herein to denote a polymeric molecule. A polymer structure can be a dendritic structure.

“Repeating unit” is used herein to denote a part of a molecule which is repeated. One example is repeated monomers which are used to build up a polymer.

“Unit” is used herein to denote a specific group of atoms in a molecule.

### DETAILED DESCRIPTION

In a first aspect there is provided a dendritic structure, said dendritic structure comprising a core and repeating units, wherein the repeating units comprise units of the type ABₓC_{y},

wherein x is 2, 3, or 4,
wherein y is 1, 2, or 3,
wherein C is selected from the group consisting of azides and alkynes, and
wherein every repeating unit is bound to at least one other unit with at least one bond selected from the group consisting of the group consisting of an ester, an amide, a thioether, an ether, a urethane, an amine, and an imine.

In one embodiment a core unit comprises more than one functional group. Examples of a core include aliphatic and aromatic units of various size. Examples of functionalities of the core include, but are not limited to —OH, —NH2, —COOH, —NCO, —CSH, and —CHO. Specific examples of cores include but are not limited to 1,1,1-tris(hydroxymethyl)propane (TMP), 1,1,1-tris(4-hydroxyphenyl)ethane (Ar), polycarbonate, polycaprolactone, poly(ethylene glycol), and di(trimethylol)propane (Di-TMP).

In one embodiment all repeating units are of the type ABₓC_{y}.

In an alternative embodiment a fraction of all repeating units are of the type ABₓC_{y}.

In one embodiment the repeating units are both of the type ABₓC_{y} and of the type ABₓ.

Thus there is the possibility to obtain dendritic structures that are made from both an ABₓC_{y} monomer and from an ABₓ monomer. As a result the interior functionality can be tailored at a specific layer or layers.

In an alternative embodiment every repeating unit is bound to at least one other unit with at least one bond selected from the group consisting of an ester, an amide, a thioether, a urethane, an imine and an ether.

In another embodiment every repeating unit is bound to at least one other unit with an ester.

In one embodiment at least one of the repeating units comprise optional spacers of length n. Example of spacers include but are not limited to alkyl chains, aromatic spacers, and hydrophilic spacers.

In one embodiment A is COOH and B is OH. In one embodiment A is COOH and B is NH2. In one embodiment A is NCO and B is OH. In one embodiment A is vinylic and B is SH. In one embodiment A is N-hydroxysuccinimide (NHS) ester and B is NH2. In one embodiment A is a halogen and B is OH.

The term vinlyic comprises allylic, acrylic and methacrylic groups.

In one embodiment B is COOH and A is OH. In one embodiment B is COOH and A is NH2. In one embodiment B is NCO and A is OH. In one embodiment B is vinylic and A is SH. In one embodiment B is N-hydroxysuccinimide (NHS) ester and A is NH2. In one embodiment B is a halogen and A is OH.

In one embodiment x is 2 or 3. In one embodiment x is 2.

In one embodiment y is 1 or 2. In one embodiment y is 1.

In one embodiment the dendritic structure is a dendrimer.

In one embodiment the dendritic structure is selected from the group consisting of a dendrimer, a dendron, a hyperbranched polymer and a dendronized polymer.

In one embodiment the dendritic structure is a dendrimer.

In one embodiment the dendritic structure is a dendron.

In one embodiment the dendritic structure is a dendritic polymer. In one embodiment the dendritic structure is a dendritic polymer comprising at least five repeating units.

In one embodiment the dendritic structure is a dendrimer with a trifunctional core, where the dendritic structure if of generation 2 or higher.

In one embodiment the dendritic structure is a dendronised polymer of generation 1 or higher.

In one embodiment the dendritic structure is a dendron of generation 3 or higher.

In one embodiment the dendritic structure is a dendritic structure of at least generation 1.

In one embodiment the dendritic structure is a dendritic structure of at least generation 2.

In one embodiment the dendritic structure is a dendritic structure of at least generation 3.

Examples of monomers according to the present invention include commercial compounds that can be transformed to ABₓC_{y} monomers. These include but are not limited tris(hydroxymethyl) aminomethane (Trizma), tris(hydroxymethyl)aminomethane hydrochloride) (Trizma*HCl), 2-(bromomethyl)-2-(methylol)-1,3-propanediol (TMP-Br) and 1,1,1-tris(hydroxymethyl)propane (TMP).

In a second aspect there is provided a method of manufacturing a dendritic structure comprising the steps

a) reacting at least two monomers of the type ABₓC_{y} with a core molecule, and
b) reacting the result from step a) with monomers of the type ABₓC_{y} to obtain a larger dendritic structure, wherein x is 2, 3, or 4,
wherein y is 1, 2, or 3, and
wherein C is selected from the group consisting of azides and alkynes.

In one embodiment the step b) is repeated. In one embodiment step b) is repeated a number of times so that a dendritic structure of the desired generation is made.

When step b) is performed once a core molecule with at least one repeating unit is obtained. Typically there are several repeating units attached to the core molecule. Examples of number of repeating units attached to a core molecule include but are not limited to 1, 2, 3, and 4.

The core molecule with directly attached repeating units is a dendritic structure of the first generation. A dendritic structure of the first generation is obtained if step b) is performed once.

If step b) is repeated, further repeating units are attached to the existing repeating units. If step b) is performed twice a dendritic structure of the second generation is obtained. If step b) is performed three times a dendritic structure of the third generation is obtained. Thus dendritic structures of different generations can be made. Examples of generations include but are not limited to 1, 2, 3, 4, 5, 6, 7, 8, 9, and 10. In one embodiment the dendritic structures are of generation 2, 3, 4, 5, or 6. In another embodiment the dendritic structures are of generation 2, 3, or 4. In another embodiment the dendritic structures are of generation 2, or 3.

In one embodiment of the method the functional group A will react with a molecule comprising at least one functional group B.

In one embodiment, the growth of the dendritic polymer structure proceeds by convergent growth. In an alternative embodiment the growth of the dendritic polymer structure proceeds by divergent growth. Thus there is provided a method wherein the repeating units are bonded with divergent growth approach. Thus there is also provided a method wherein the repeating units are bonded with convergent growth approach.

In one embodiment the manufactured dendritic polymer structures will have groups B predominantly in the outer layer and groups C predominantly in the interior and to a lesser extent in the outer layer.

Both the groups B and the groups C are available for functionalization after the manufacture of the dendritic polymer structure.

In a third aspect there is provided a method of adding functional groups to a dendritic structure, wherein a functional group is added to a group C in the dendritic structure. Group C is selected from azides and alkynes.

In one embodiment at least two different groups are added in one step to a group C and a group B respectively. One type of functional groups is added to a group C and a different type of functional groups is added to a group B simultaneous in one step. This is possible because the chemical reactions are orthogonal, that is they do not interfere with each other, or they only interfere with each other to a very low extent.

Examples of functional groups which can be added to the dendritic structure include but are not limited to: hydrophilic groups, hydrophobic groups, crystalline groups, dyes, fluorescent dyes, carbohydrates, active drugs such as antiviral peptides, antifungal peptides, antibacterial peptides, anticancer peptides, cathelicidin, bacteriocins, bacteriophages, antimicrobial agents, beta-lactams, penicillins, cephalosporins, penicillin G, cephalothin, semisynthetic penicillin, ampicillin, amoxycillin, clavulanic acid, clavamox, monobactams aztreonam, carboxypenems imipenem, aminoglycosides streptomycin, gentamicin, glycopeptides vancomycin, lincomycins clindamycin, macrolides, erythromycin, polymyxin, bacitracin, polyenes, amphotericin, nystatin, rifamycins, rifampicin, tetracyclines, semisynthetic tetracycline, doxycycline, chloramphenicol, pyrazinamide, sulfa drugs, sulfonamide, antiseptic agents, chlorhexidine, iodine/iodophors, triclosan, quaternary ammonium compounds, phosphate imidazolinium compounds, dimethyl benzyl ammonium chloride compounds, dimethyl ethylbenzyl ammonium chloride, alkyl dimethyl ammonium chloride, paradiisobutylphenoxyethoxyethyl dimethyl benzyl ammonium chloride, poly(hexamethylene biguanide hydrochloride), and tetramine compounds. Further examples include but are not limited to essential oils such as oregano oil, tea tree oil (melaleuca Oil), mint oil, sandalwood oil, clove oil, nigella sativa (black cumin) oil, onion oil (allium cepe)—phytoncides, leleshwa oil, lavender oil, lemon oil, eucalyptus oil, peppermint oil, and cinnamon oil. Further examples include but are not limited to nitrofuranes such as nitrofurantoin and nitrofurazone. Further examples include but are not limited to antithrombogenic substances such as heparin group (platelet aggregation inhibitors), methacryloyloxyethyl phosphorylcholine polymer, polyphloretinphosphate, heparin, heparan sulphate, hirudin, lepirudin, dabigatran, bivalirudin, fondaparinux, ximelagatran, direct thrombin inhibitors, argatroban, melagatran, ximelagatran, desirudin, defibrotide, dermatan sulfate, fondaparinux, rivaroxaban, antithrombin III, bemiparin, dalteparin, danaparoid, enoxaparin, nadroparin, parnaparin, reviparin, sulodexide, tinzaparin, vitamin K antagonists, acenocoumarol, clorindione, dicumarol (dicoumarol), diphenadione, ethyl biscoumacetate, phenprocoumon, phenindione, tioclomarol, warfarin, platelet aggregation inhibitors, abciximab, acetylsalicylic acid (aspirin), aloxiprin, beraprost, ditazole, carbasalate calcium, cloricromen, clopidogrel, dipyridamole, eptifibatide, indobufen, iloprost, picotamide, prasugrel, prostacyclin, ticlopidine, tirofiban, treprostinil, triflusal, enzymes, alteplase, ancrod, anistreplase, brinase, drotrecogin alfa, fibrinolysin, protein C, reteplase, saruplase, streptokinase, tenecteplase, urokinase, chelators, citrate, EDTA, and oxalate. Further examples include but are not limited to anti-inflammatory substances, non-steroidal anti-inflammatory drugs, salicylates (such as aspirin (acetylsalicylic acid), diflunisal, ethenzamide), arylalkanoic acids (such as diclofenac, indometacin, sulindac), 2-arylpropionic acids (profens) (such as carprofen, flurbiprofen, ibuprofen, ketoprofen, ketorolac, loxoprofen, naproxen, tiaprofenic acid), N-arylanthranilic acids (fenamic acids) (such as mefenamic acid), pyrazolidine derivatives (such as phenylbutazone), oxicams (such as meloxicam, piroxicam), coxibs (such as celecoxib, etoricoxib, parecoxib, rofecoxib, valdecoxib), sulphonanilides (such as nimesulide), diclofenac, flurbiprofen, ibuprofen, indometacin, ketoprofen, naproxen, piroxicam, and eicosanoids. Further examples include but are not limited to any of a group of substances that are derived from arachidonic acid, including leukotrienes, thromboxanes, and prostaglandins. Further examples include but are not limited to immunosuppressive drugs. Further examples include but are not limited to analogues of rapamycin, such as tacrolimus, sirolimus and everolimus, paclitaxel, docetaxel, and erlotinib.

In one embodiment the functionalization of both groups B and C is simultaneous to yield the final product in a one-pot synthesis including in-situ reactions.

In a fourth aspect there is provided a dendritic structure further comprising at least one functional group, wherein the at least one functional group is attached to an azide or alkyne in the dendrimer molecule. The group C is selected from azides and alkynes and those groups serve as groups where various functional groups can be attached.

In a fifth aspect there is provided use of a dendritic structure within at least one area selected from the group consisting of drug delivery systems, tissue engineering, data storage devices, markers for imaging, diagnostics, vaccines, phototherapeutics, optical devices, semiconductor, bioactive hydrogels and catalysts.

The use of dendritic polymer materials is described in more detail in the following twelve references which are explicitly incorporated herein by reference in their entirety. The twelve references below describe use of dendritic structures within various areas. The novel dendritic structures according to the present invention can be used as described in these references. Use within drug delivery systems, diagnostics, vaccines, phototherapeutics, optical devices and tissue engineering are described in references 1-6. Use within data storage devices is described in references 7-8. Use as markers for imaging is described in references 1-6. Use as a semiconductor is described in reference 9. Use as bioactive hydrogels is described in reference 10. Use as catalysts is described in references 11-12.
(1) C. C. Lee, J. A. MacKay, J. M. J. Fréchet and F. C. Szoka, Nature Biotechnology, 2005, 23 (12), 1517-1526
(2) B. Helms and E. W. Meijer, Science (Washington, D.C., United States), 2006, 313 (5789), 929-930
(3) U. Boas, J. B. Christensen and P. M. H. Heegaard, 2006, Dendrimers in Medicine and Biotechnology: New Molecular Tools 179 pp, RSC publishing, ISBN-10: 0-85404-852-9.
(4) S. Svenson and D. A. Tomalia, Advanced Drug Delivery Reviews, 2005, 57 (15), 2106-2129
(5) J. Frechet and D. Tomalia, 2001, Dendrimers and Other Dendritic Polymers 800 pp, John Wiley & Sons, Ltd.
(6) S.-E. Stiriba, H. Frey and R. Haag, Angewandte Chemie, International Edition, 2002, 41 (8), 1329-1334
(7) B. Lohse, R. Vestberg, M. T. Ivanov, S. Hvilsted, R. H. Berg, P. S. Ramanujam and C. J. Hawker, Journal of Polymer Science, Part A: Polymer Chemistry, 2007, 45 (19), 4401-4412
(8) A. Archut, F. Vogtle, L. De Cola, G. C. Azzellini, V. Balzani, P. S. Ramanujam and R. H. Berg, Chemistry—A European Journal, 1998, 4 (4), 699-706
(9) C. J. Hawker, J. L. Hedrick, R. D. Miller and W. Volksen, MRS Bulletin, 2000, 25 (4), 54-58
(10) M. W. Grinstaff, Journal of Polymer Science, Part A: Polymer Chemistry, 2007, 46 (2), 383-400
(11) C. Hajji and R. Haag, Topics in Organometallic Chemistry, 2006, 20 (Dendrimer Catalysis), 149-176
(12) R. Haag and S. Roller, Polymeric Materials in Organic Synthesis and Catalysis, 2003, 305-344

Further examples of use of the present dendritic structures include, but are not limited to use in:
Coatings—including phenolic resin, amino resins, polyurethane resins (foam or elastomer), (meth)acrylic resins epoxy resin, silicone-containing resins and unsaturated vinylic resins including (meth)acrylic, allylic, maleimide, norbornene functional resins.
Hydrogels including PEG, PPO, poly(acrylic acid), poly(hydroxyethyl methacrylate), poly(hydroxyethyl acrylate) poly(dimethyl acrylamide), polyacrylamide, poly(4-vinyl pyridine), polyNIPAM and other water soluble polymer
Polyfunctional initiators for controlled radical polymerization (CRP) or living free-radical polymerization (LFRP) techniques (including atom transfer radical polymerization (ATRP), reversible addition-fragmentation chain transfer (RAFT), nitroxide mediated polymerization (NMP)), ring-opening metathesis polymerization (ROMP), ring-opening polymerization (ROP)
Dental and other composite materials
Additives to control viscosity, rheology, solubility, stabilizing capacity etc
Dendronized polymers using CRP or LFRP techniques including ATRP, RAFT, NMP, ROMP and ROP,
Dendritic-linear polymer,
Hybrid organic-inorganic materials
Water soluble materials (PEG, PPO, poly(acrylic acid), poly(HEMA), poly(dimethyl acrylamide), poly(acryl amide), poly(NiPAAM), poly(4-vinyl pyridine) or other watersoluble polymers),
Organosoluble polymer (acrylate based polymer, methacrylic based polymer, styrene based polymers, triazole based polymers, PCL, PLA and other biocompatible polymers and other hydrophobic polymers),
Amphiphilic architectures by a combination of the building blocks mentioned previously to tailor features such as solubility (water, organic solvents etc)

Still further examples of use of the present dendritic structures include but are not limited to the following material science applications:
Well-defined building blocks for precise architectural control.
Dendrimers forming nanoparticles, including intra- and/or intermolecular collapse.
Deposition of dendrimers on solid surfaces by Layer-by-Layer deposition or microcontact printing.
Dispersing agents for nanoparticles, including Silver, Gold, GaSe, SiOₓ, TiO₂,
Dendrimer vehicles for encapsulation and/or delivery (vaccines, drugs, prodrugs, genes, cancer therapeutics, fragrances, proteins, enzymes, affibody, peptide, DNA, RNA etc) where especially the feature of multivalency is taken into account, both to incorporate two or more active compounds but also making use of the numerous functionalities to accomplish high-loading capacity.
Dendrimer scaffolds containing growth factors, co-factors etc for skin, tissue and bone repair,
Dendrimer scaffolds where the dendrimer skeleton comprises optically active compounds (non-linear optics—optical power limiting, imaging, MRI, PET, bioimaging, fluorescence, multifunctional probes)
Dendrimer scaffolds comprising conducting moieties to result in conducting polymers.

In a sixth aspect there is provided a method for the manufacture of a particle comprising a crosslinking reaction of a dendritic structure. In one embodiment azide groups in a dendritic structure react to form a nitrene group. Under dilute conditions the intramolecular cross linking is favored. Under concentrated conditions the intermolecular cross linking is favored. If intramolecular crosslinking is desired dilute conditions should be used. In one embodiment the intermolecular collapse is minimized at a concentration of 0.5 mg dendrimer per 1 ml solvent and below.

In a seventh aspect there is provided a particle manufactured from a dendritic structure.

Examples of use of the particles include but are not limited to encapsulation of low molecular compounds such as potent drugs, chelating species and fluorescent dyes.

In an eight aspect there is provided a hydrogel manufactured from a dendritic structure. In one embodiment these gels include reservoirs of active groups with the capability to trigger chemical or biological activity.

Other features and uses of the invention and their associated advantages will be evident to a person skilled in the art upon reading the description and the examples.

It is to be understood that this invention is not limited to the particular embodiments shown here. The following examples are provided for illustrative purposes and are not intended to limit the scope of the invention since the scope of the present invention is limited only by the appended claims and equivalents thereof.

### EXAMPLES

A variety of dendritic structures were synthesized based on different cores and different monomer composition. This was done to elucidate the wide variety and functionalities that can be obtained by using ABₓC_{y}-monomers.

### Nomenclature: A Selection of Compounds are Given Below

### Acet=Acetylene

### Ac=Acetonide

### N₃=Azide

Acid=Carboxylic acid
Ar=1,1,1-tris(4-hydroxyphenyl)ethane
TMP=1,1,1-tris(hydroxymethyl)propane
di-TMP=di(trimethylol)propane
DMAP=dimethylaminopyridine
DPTS=4-(dimethylamino)pyridium p-toluenesulfonate
Trizma=tris(hydroxymethyl)aminomethane
Trizma*HCl=tris(hydroxymethyl)aminomethane hydrochloride
DCC=N,N′-dicyclohexylcarbodiimide
TMP-Br=2-(bromomethyl)-2-(methylol)-1,3-propanediol

### Summary of the Examples

### Dendrimer Synthesis Based on ABₓC_{y} Monomers

### Example 1-2

### Synthesis of Dendrimers with Active Hydroxyl Groups at the Chain End and Active Acetylenes in the Interior

### Example 1

Using TMP as a B₃ core.
Trizma*HCl as a precursor for the development of AB₂C monomer, compound 5.
DCC as an esterification agent for the dendritic growth
Acidic conditions for activation of each dendritic scaffold
Growth via the divergent growth approach.

### Example 2

Using Ar as a B₃ core.
Trizma*HCl as a precursor for the development of AB₂C monomer, compound 5.
DCC as an esterification agent for the dendritic growth
Acidic conditions for activation of each dendritic scaffold
Growth via the divergent growth approach.

### Example 3

### Synthesis of Dendrimers with Active Hydroxyl Groups at the Chain End and Active Azide in the Interior

Using TMP as a B₃ core.
TMP-Br as a precursor for the development of AB₂C monomer.
DCC as an esterification agent for the growth
Acidic conditions for activation of each dendritic scaffold
Growth via the divergent growth approach.

### Example 4

Using Ar as a B₃ core.
Trizma*HCl as a precursor for the development of AB₂C monomer with pendant azide as C.
Anhydride activation of AB₂Cₓ for the dendritic growth
Acidic conditions for activation of each dendritic scaffold
Growth via the divergent growth approach.

### Example 5

Using TMP as a B₃ core.
TMP-BR as a precursor for the development of AB₂C monomer with pendant acetylenes as C.
Anhydride activation of AB₂Cₓ for the dendritic growth
Acidic conditions for activation of each dendritic scaffold
Growth via the divergent growth approach.

### Example 6

Using TMP as a B₃ core.
TMP-BR as a precursor for the development of AB₂C monomer with pendant acetylenes as C.
Anhydride activation of AB₂Cₓ for the dendritic growth
Acidic conditions for activation of each dendritic scaffold

Growth via the divergent growth approach

### Structure and Functionality

In the table below there is summarised a list of synthesized dendrimers with different core, interior and peripheral functionality. There is the dendrimer number (Dendrimer), generation (Gen), number of N₃ groups (N₃), number of acetylene groups (Acetylene), number of acetonide groups (Acetonide) and number of OH groups (OH).

| Dendrimer | Gen | N₃ | Acetylene | Acetonide | OH |
|---|---|---|---|---|---|
| 6 | 1 | | 3 | 3 | |
| 7 | 1 | | 3 | | 6 |
| 8 | 2 | | 9 | 6 | |
| 9 | 2 | | 9 | | 12 |
| 10 | 3 | | 21 | 12 | |
| 11 | 1 | | 3 | 3 | |
| 12 | 1 | | 3 | | 6 |
| 13 | 2 | | 9 | 6 | |
| 14 | 1 | | 4 | 4 | |
| 15 | 1 | | 4 | | 6 |
| 19 | 1 | 3 | | 3 | |
| 20 | 1 | 3 | | | 6 |
| 21 | 2 | 9 | | 6 | |
| 22 | 2 | 9 | | | 12 |
| 23 | 3 | 21 | | 12 | |

### Experimental

### Instrumentation

### MALDI-TOF

THF/DHB/Na⁺-matrix and THF/9-nitroanthracene/Na⁺-matrix were used for sample preparation for MALDI-TOF analysis, concentration 1 mg/ml of sample in THF (40□l Matrix solution/5□l sample solution). The MALDI-TOF MS spectrum acquisitions were conducted on a Bruker UltraFlex MALDI-TOF MS with SCOUT-MTP Ion Source (Bruker Daltonics, Bremen) equipped with a N₂-laser (337 nm), a gridless ion source and reflector design. All spectra were acquired using a reflector-positive method with an acceleration voltage of 25 kV and a reflector voltage of 26.3 kV. The detector mass range was set to 500-10000 Da in order to exclude high intensity peaks from the lower mass range. The laser intensity was set to the lowest value possible to acquire high resolution spectra. The obtained spectra were analyzed with FlexAnalysis Bruker Daltonics, Bremen, version 2.2.

### Size Exclusion Chromatography (SEC)

SEC using THF (1.0 mL min⁻¹) as the mobile phase was performed at 35° C. using a Viscotek TDA model 301 equipped with two GMH_{HR}-M columns with TSK-gel (mixed bed, MW resolving range: 300-100 000 g/mol) from Tosoh Biosep, a VE 5200 GPC autosampler, a VE 1121 GPC solvent pump, and a VE 5710 GPC degasser (all from Viscotek corp.). A calibration method was created using narrow linear polystyrenes standards. Corrections for the flow rate fluctuations were made using toluene as an internal standard. Viscotek OmniSEC version 4.0 software was used to process data.

### ¹H NMR and ¹³C NMR

NMR experiments were performed on a Bruker Avance 400 MHz NMR instrument. Proton NMR spectra were acquired with a spectral window of 20 ppm, an acquisition time of 4 seconds, a relaxation delay of 1 second. ¹³C NMR spectra were acquired with a spectral window of 240 ppm, an acquisition time of 0.7 seconds, a relaxation delay of 2 seconds. The spectra were calibrated with respect to the solvent peak?

### Flash Chromatography

was performed using 30-60 μm, 60 Å silica gel from Sigma-Aldrich.

### General Procedure for Deprotection of the Acetonide Group.

All acetonide containing compounds were dissolved in MeOH in a flask and heated to 45° C. without the use of a stopper. An acidic catalyst or resin, such as DOWEX® 50W-X2, was added to each solution and the deprotections were monitored by TLC. The full conversion of the acetonide to hydroxyl groups was confirmed with MALDI-TOF and ¹H and ¹³C NMR. The acidic resin was filtered off and the filtrate was concentrated by evaporation of the solvent.

### General Procedure for Addition of COOH-Acet-Ac (5) or COOH—N₃-Ac (18) to a Dendrimer Using DCC-Coupling.

The hydroxyl functional dendrimer was dissolved in DCM followed by the addition of monomer (1.2 eqv./OH), DMAP (0.1 eqv./OH), DPTS (0.2 eqv./OH) and DCC (1.2 eqv./OH). The reaction was kept over night at room temperature. The DCC-complex was filtered off and the obtained crude product was purified by flash chromatography.

### Synthesis of Ester Based Acetylene Functional Dendrimers Based on ABₓC_{y} Monomer

### I. Synthesis of the ABₓCₓ Monomer in which

### A is —COOH

### B is —OH

### C is —CCH

x is 2
y is 1

### Synthesis of Acetylene Acid (1)

Propargyl alcohol (40.0 g, 0.741 mol), DMAP (17.43 g, 0.143 mol) and succinic anhydride (85.7 g, 0.856 mol) were dissolved in DCM (100 ml) and left to react over night. Water (75 ml) was added to the solution followed by extraction with NaHSO₄ (10%) 3 times. The organic phase was then dried with MgSO₄, filtered and concentrated. Yield: 82% (107.6 g).

¹H-NMR (CDCl₃, 400 MHz), δ 2.48 (t, J=2.4, 1H, —CH), 2.62-2.70 (m, 4H, —CH₂C═O—), 4.67 (d, J=2.4 Hz, 2H, —OCH₂CCH) ppm,

¹³C-NMR (CDCl₃, 400 MHz) δ 28.46, 28.67, 52.23, 75.05, 77.27, 171.32, 178.19.

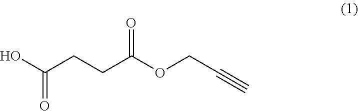

### Synthesis of Acetonide Protected Trizma® (2)

Trizma hydrochloride (100 g, 0.634 mol), toulene-4-sulfonic acid monohydrate (5.94 g, 0.0312 mol) and 2,2-dimethoxypropane (99.1 g, 0.951 mol) were dissolved in DMF and left over night. TEA (8 ml) was then added to the solution to neutralise p-TSA followed by concentration. The concentrated mixture was precipitated in EtOAc and filtrated. 50 ml of TEA was added to the filtrate and a second filtration was performed. The filtrate was then concentrated again and a second precipitation was prepared in cold diethylether. The product was collected as a white powder. Yield 83% (84.7 g).

¹H-NMR (CDCl₃, 400 MHz), δ 1.27 (s, 3H, —CH₃), 1.35 (s, 3H, —CH₃), 3.35 (s, 2H, —CH₂OH), 3.57 (d, 2H, J=11 Hz, —CH₂O—), 3.41 (d, 2H, J=11 Hz, —CH₂O—) ppm.

¹³C-NMR (CDCl₃, MHz) δ 21.61, 25.50, 49.11, 63.42, 65.88, 97.01.

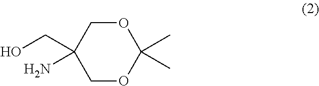

### Synthesis of Acetylene Anhydride (3)

Compound 1 (80.0 g, 0.513 mol) was dissolved in DCM (150 ml) and cooled to 0° C. followed by addition DCC (52.9 g, 0.256 mol). The reaction was left to reach room temperature over night and then filtered. The colourless product was obtained as white solid after concentration. Yield: 79% (59.6 g).

¹H-NMR (CDCl₃, 400 MHz), δ 2.49 (t, 2H, —CH), 2.70-2.83 (m, 8H, —CH₂C═O—), 4.70 (d, J=2.1 Hz, 4H, —OCH₂C—) ppm.

¹³C-NMR (CDCl₃, MHz) δ 28.23, 30.01, 52.40, 75.16, 77.23, 167.57, 170.83.

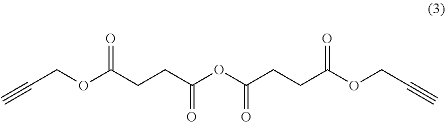 [SMILES: C#CCOC(=O)CCC(=O)OC(=O)CCC(=O)OCC#C]

### Synthesis of Acetylene-Ac-OH (4)

Compound 2 (55.0 g, 0.187 mol), TEA (28.3 g, 0.281 mol) and 3 (49.5 g, 168 mol) were dissolved in DCM (400 ml) at 0° C. The reaction was monitored with ¹³C-NMR. The crude solution was extracted with NaHSO₄ and concentrated. The product was purified by flash chromatography eluting the product in 45/55 EtOAc/Heptane. The product was obtained as white powder after removal of solvent. Yield 80% (44.7 g).

¹H-NMR (CDCl₃, 400 MHz) δ 1.42 (s, 6H, —CH₃), 2.46 (t, 1H, —CH), 2.57 (t, 2H, —CH₂COO—), 2.71 (t, 2H, —CH₂CON—), 3.66 (s, 2H, —CH₂OH), 3.78-3.85 (m, 4H, —OCH₂—), 4.68 (d, J=2.4, 2H, —OCH₂CCH), 6.34 (s, 1H, —NH) ppm.

¹³C-NMR (CDCl₃, MHz) δ 19.42, 27.57, 29.26, 31.24, 52.29, 55.18, 63.97, 64.25, 75.05, 77.40, 98.89, 171.96, 172.47.

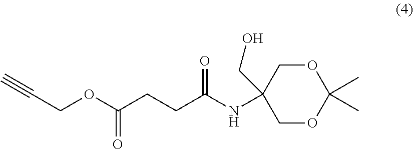 [SMILES: C#CCOC(=O)CCC(=O)NC1(CO)COC(C)(C)OC1]

### Synthesis of Acid Functional Acetonide Protected Trizma-Acetylene (5)

The acetonide protected Trizma® derivative 4 (41.4 g, 0.138 mol) and DMAP was dissolved in DCM (100 ml). Succinic anhydride (16.8 g, 0.166 mol) were dissolved in DCM (100 ml) and added to the solution. The reaction was left over night followed by quenching of the anhydride by water (75 ml). The organic phase was extracted with 10 wt % NaHSO₄ in H₂0, dried with MgSO₄ and then concentrated. Yield was 87% (47.9 g).

¹H-NMR (CDCl₃, 400 MHz) δ 1.39 (d, J=12 Hz, 6H, —CH₃), 2.49 (m, 3H, —CH and —CH₂C—), δ 2.68 (m, 6H, —CH₂C—), 3.73 (d, J=12.0 Hz, 2H, —CH₂O—), 4.29 (d, J=12.0 Hz, 2H, —CH₂O—), 4.49 (s, 2H, —OCH₂—), 4.69 (d, J=2.4, 2H, —OCH₂CCH), 6.03 (s, 1H, —NH) ppm.

¹³C-NMR (CDCl₃, MHz) δ 13.97, 20.36, 20.83, 26.30, 29.04, 30.89, 52.05, 54.81, 60.20, 63.50, 63.60, 74.98, 7730, 98.58, 170.99, 171.84, 172.34.

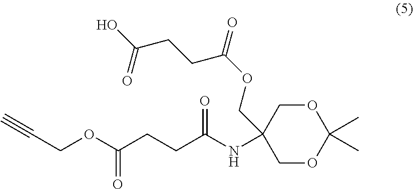 [SMILES: C#CCOC(=O)CCC(=O)NC1(COC(=O)CCC(=O)O)COC(C)(C)OC1]

### II. Typical Growth of an Ester Based Acetylene Functional Dendrimer Using TMP Core Unit

### Synthesis of Dendrimer TMP-G1-(Acet)₃-(Ac)₃ (6)

Compound 5 (10.7 g, 26.8 mmol), TMP (1.00 g, 7.45 mmol), DMAP (0.27 g, 2.2 mmol) and DPTS (1.29 g, 4.4 mmol) were dissolved in anhydrous DCM. DCC (5.52 g, 26.8 mmol) was added to the 0° C. solution. The reaction was left over night and then analyzed using MALDI-TOF to make sure that fully substitution of hydroxyl groups had occurred. The slurry was filtered of, extracted with NaHSO₄ and then concentrated. The crude oil was purified by flash chromatography eluting the product in 70/30-EtOAc/heptane. The product was obtained as a colourless oil after removal of solvent. Yield was 95% (9.0 g).

¹H-NMR (CDCl₃, 400 MHz) δ 0.88 (t, 3H, —CH₃), 1.40 (s, 9H, —CH₃), 1.49 (m, 11H, —CH₂C— and —CH₃), 2.49 (m, 9H, —CCH, —CH₂CH₂C—), 2.67 (m, 18H, —CH₂CH₂C—), 3.76 (d, 6H, J=12.0 Hz, —CH₂O—), 4.02 (s, 6H, —CH₂C—, core), 4.24 (d, 6H, J=12.0 Hz, —CH₂O—), 4.49 (s, 6H, —CH₂O—), 4.68 (d, 6H, J=2.4 Hz, —OCH₂CCH—), 6.04 (s, 6H, —CONH—) ppm.

¹³C-NMR (CDCl₃, MHz) δ 7.28, 22.8, 24.26, 28.86, 28.89, 31.02, 40.71, 52.11, 52.96, 62.19, 63.64, 63.90, 75.00, 77.54, 98.66, 171.63, 171.96, 172.10, 172.31.

MALDI: Calc. [M_{W}+Na⁺]=1300.52 g/mol, Found [M_{W}+Na⁺]=1300.59 g/mol.

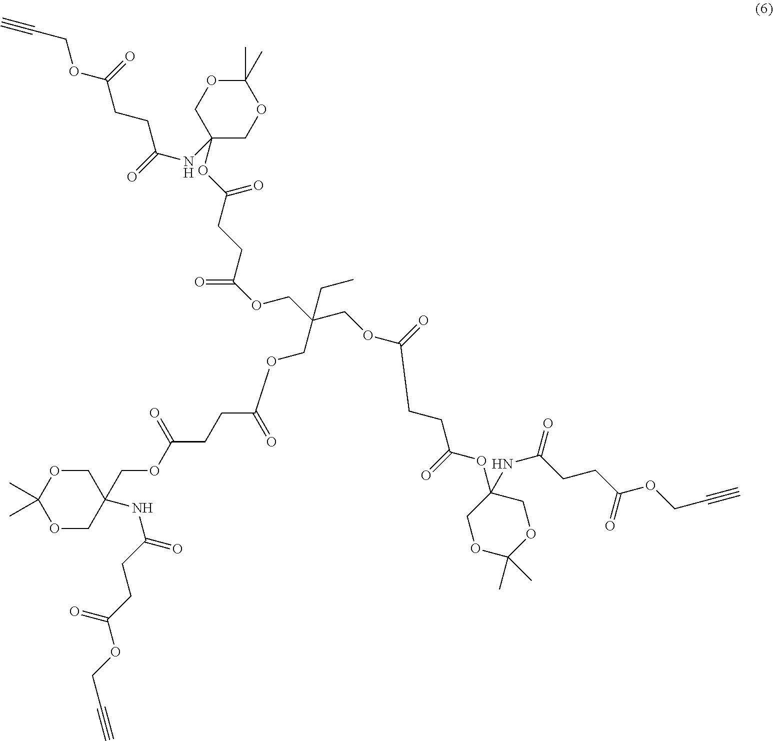 [SMILES: C#CCOC(=O)CCC(=O)NC1(COC(=O)CCC(=O)OCC(CC)(COC(=O)CCC(=O)OC2(NC(=O)CCC(=O)OCC#C)COC(C)(C)OC2)COC(=O)CCC(=O)OC2(NC(=O)CCC(=O)OCC#C)COC(C)(C)OC2)COC(C)(C)OC1]

### Synthesis of Dendrimer TMP-G1-(Acet)₃-(OH)₆ (7)

Dendrimer 6 (7.00 g, 5.48 mmol) was dissolved in methanol (150 ml) and heated to 45° C. followed by addition of 10 g DOWEX® 50W-X2. The reaction was monitored with TLC and MALDI-TOF. The product was purified by flash chromatography eluting the product in 4/96-methanol/EtOAc. A colourless oil was obtained after removal of solvent. Yield 91% (5.8 g).

¹H-NMR (CDCl₃, 400 MHz) δ 0.89 (t, J=7.4 Hz, 3H) δ 1.40 (s, CCH₃, 9H), δ 1.47 (q, J=7.4, —CH₂CH₃, 2H), δ 1.49 (s, CCH₃, 9H), δ 2.48-2.69 (m, —CCH, —CH₂CH₂—, 27H), δ 3.78 (d, J=12.0, —CCH₂O—, 6H), δ 4.02 (s, —OCH₂C—, 6H), δ 4.25 (d, J=12.0, —CCH₂O—, 6H), δ 4.49 (s, —OCH₂C—, 6H), δ 4.68 (d, J=2.46, 6H), δ 6.05 (s, NH, 3H) ppm.

¹³C-NMR (CDCl₃, MHz) δ 7.28, 22.80, 24.26, 28.86, 28.89, 28.96, 31.02, 40.71, 52.11, 52.96, 62.19, 63.64, 63.90, 75.00, 77.54, 98.66, 171.63, 171.96, 171.10, 171.31 ppm.

MALDI: Calc. [M_{W}+Na⁺]=1180.43 g/mol, Found [M_{W}+Na⁺]=1180.46 g/mol.

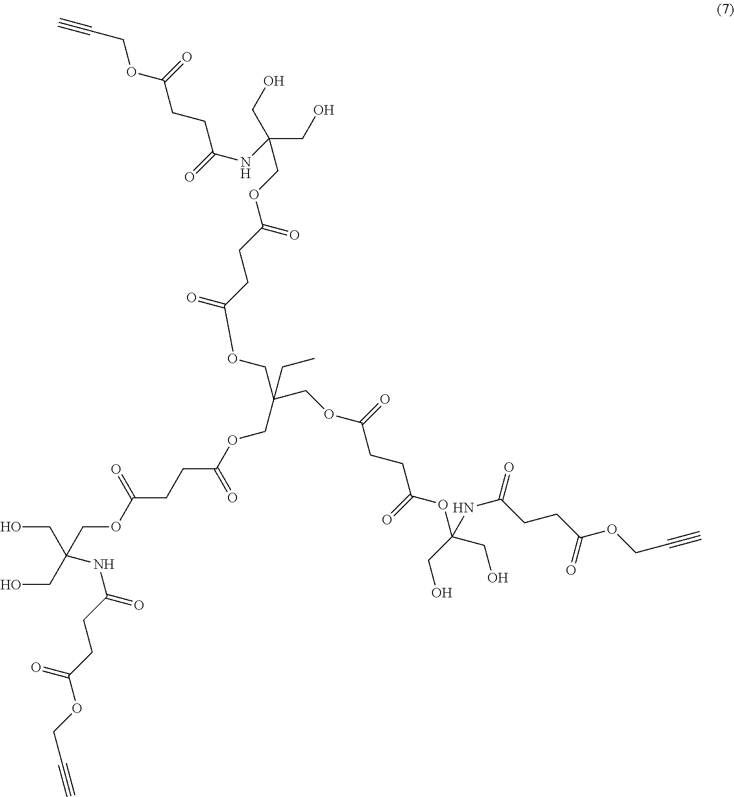 [SMILES: C#CCOC(=O)CCC(=O)NC(CO)(CO)COC(=O)CCC(=O)OCC(CC)(COC(=O)CCC(=O)OCC(CO)(CO)NC(=O)CCC(=O)OCC#C)COC(=O)CCC(=O)OC(CO)(CO)NC(=O)CCC(=O)OCC#C]

### Synthesis of Dendrimer TMP-G2-(Acet)₉-(Ac)₆ (8)

Compound 5 (7.44 g, 18.6 mmol), 7 (3.00 g, 2.59 mmol), DMAP (0.19 g, 1.55 mmol) and DPTS (0.91 g, 3.1 mmol) were dissolved in anhydrous DCM. DCC (3.83 g, 18.6 mmol) was added to the 0° C. solution. The reaction was left over night and then analyzed using MALDI-TOF to make sure that fully substitution of hydroxyl groups had occurred. The slurry was filtered of, extracted with NaHSO₄ and then concentrated. The crude oil was purified by flash chromatography eluting the product in 5/95:MeOH/EtOAc. The product was obtained as a colourless oil after removal of solvent. Yield was 87% (7.8 g).

¹H-NMR (CDCl₃, 400 MHz) δ 0.88 (t, 3H, —CH₃), 1.40 (s, 18H, —CH₃), 1.47 (m, 20H, —CH₂C— and —CH₃), 2.51 (m, 24H, —CCH, —CH₂CH₂C—), 2.67 (m, 54H, —CH₂CH₂C—), 3.78 (d, 12H, J=12.0 Hz, —CH₂O—), 4.03 (s, 6H, —CH₂C—, core), 4.22 (d, 12H, J=12.0 Hz, —CH₂O—), 4.42 (s, 16H, —CH₂O— and —OCH₂—), 4.48 (s, 12H, —CH₂O—), 4.67 (d, 6H, J=2.4 Hz, —OCH₂CCH—), 4.68 (d, 12H, J=2.4 Hz, —OCH₂CCH—), 6.10 (s, 6H, —CONH—) and 6.29 (s, 3H, —CONH—) ppm.

¹³C-NMR (CDCl₃, MHz) δ 7.32, 22.79, 24.27, 28.75, 28.91, 31.00, 40.67, 52.11, 52.87, 57.83, 60.36, 62.26, 62.60, 63.66, 63.96, 75.07, 75.15, 77.21, 77.59, 98.67, 171.69, 171.84, 171.98, 172.03, 172.43 ppm.

MALDI: Calc. [M_{W}+Na⁺]=3467.28 g/mol, Found [M_{W}+Na⁺]=3468.38 g/mol.

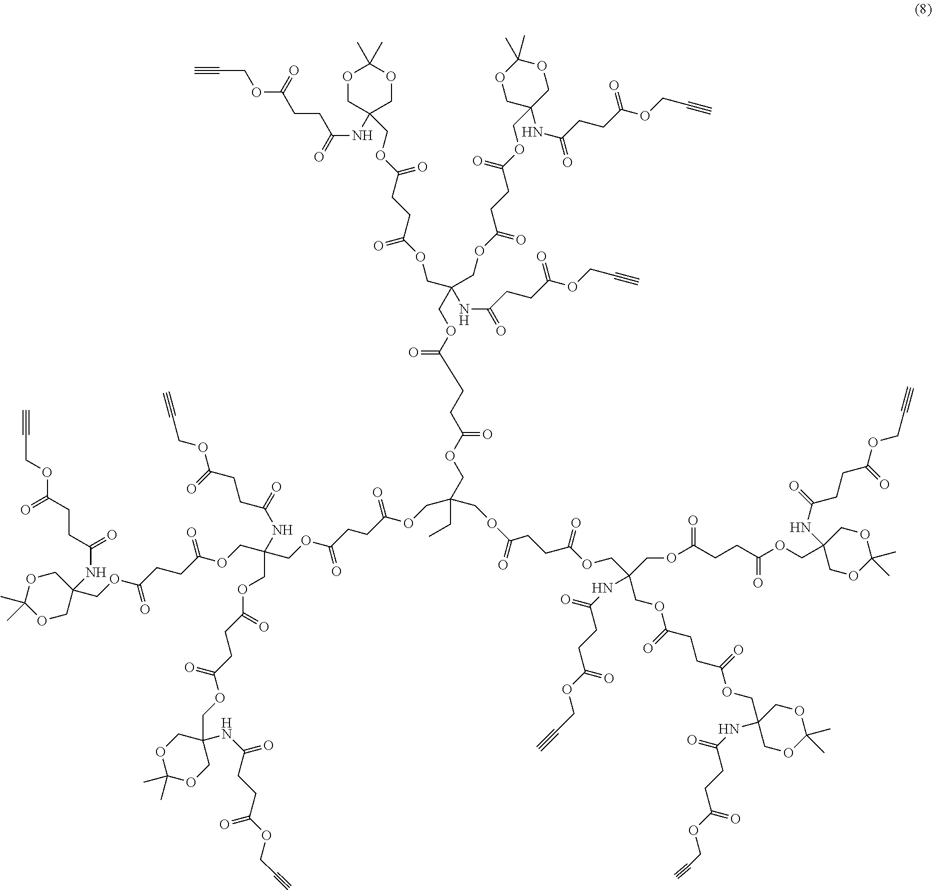 [SMILES: C#CCOC(=O)CCC(=O)NC(COC(=O)CCC(=O)OCC(CC)(COC(=O)CCC(=O)OCC(COC(=O)CCC(=O)OCC1(NC(=O)CCC(=O)OCC#C)COC(C)(C)OC1)(COC(=O)CCC(=O)OCC1(NC(=O)CCC(=O)OCC#C)COC(C)(C)OC1)NC(=O)CCC(=O)OCC#C)COC(=O)CCC(=O)OCC(COC(=O)CCC(=O)OCC1(NC(=O)CCC(=O)OCC#C)COC(C)(C)OC1)(COC(=O)CCC(=O)OCC1(NC(=O)CCC(=O)OCC#C)COC(C)(C)OC1)NC(=O)CCC(=O)OCC#C)(COC(=O)CCC(=O)OCC1(NC(=O)CCC(=O)OCC#C)COC(C)(C)OC1)COC(=O)CCC(=O)OCC1(NC(=O)CCC(=O)OCC#C)COC(C)(C)OC1]

### Synthesis of Dendrimer TMP-G2-(Acet)₉-(OH)₁₂ (9)

Dendrimer 8 (5 g, 1.45 mmol) was dissolved in methanol (150 ml) and heated to 45° C. followed by addition of 10 g Dowex. The reaction was monitored with TLC and MALDI-TOF. The product was purified by flash chromatography eluting the product in 10/90-methanol/EtOAc. A colourless oil was obtained after removal of solvent. Yield 94% (4.4 g).

¹H-NMR (DMSO-d₆, 400 MHz) δ 0.81 (t, 3H, —CH₃), 1.49 (q, 2H, —CH₂—, core), 2.42-2.66 (m, 72H, —CH₂CH₂C—), 3.46 (s, 9H, —CCH), 3.55-3.59 (s, 24H, —CH₂OH), 3.96 (s, 6H, —CH₂CO—, core), 4.19-4.28 (m, 30H, —CH₂O—, G#1 and G#2), 4.66 (s, 6H, —OCH₂CCH—), 4.69 (s, 12H, —OCH₂CCH—) ppm.

¹³C-NMR (DMSO-d₆, MHz) δ 7.68, 28.76, 28.90, 29.12, 30.55, 47.34, 47.81, 48.23, 51.83, 60.44, 60.72, 60.80, 62.25, 75.09, 77.81, 78.83, 171.77, 171.91, 171.98, 172.11, 172.19 and 172.88 ppm.

MALDI: Calc. [M_{W}+Na⁺]=3227.09 g/mol, Found [M_{W}+Na⁺]=3228.49 g/mol.

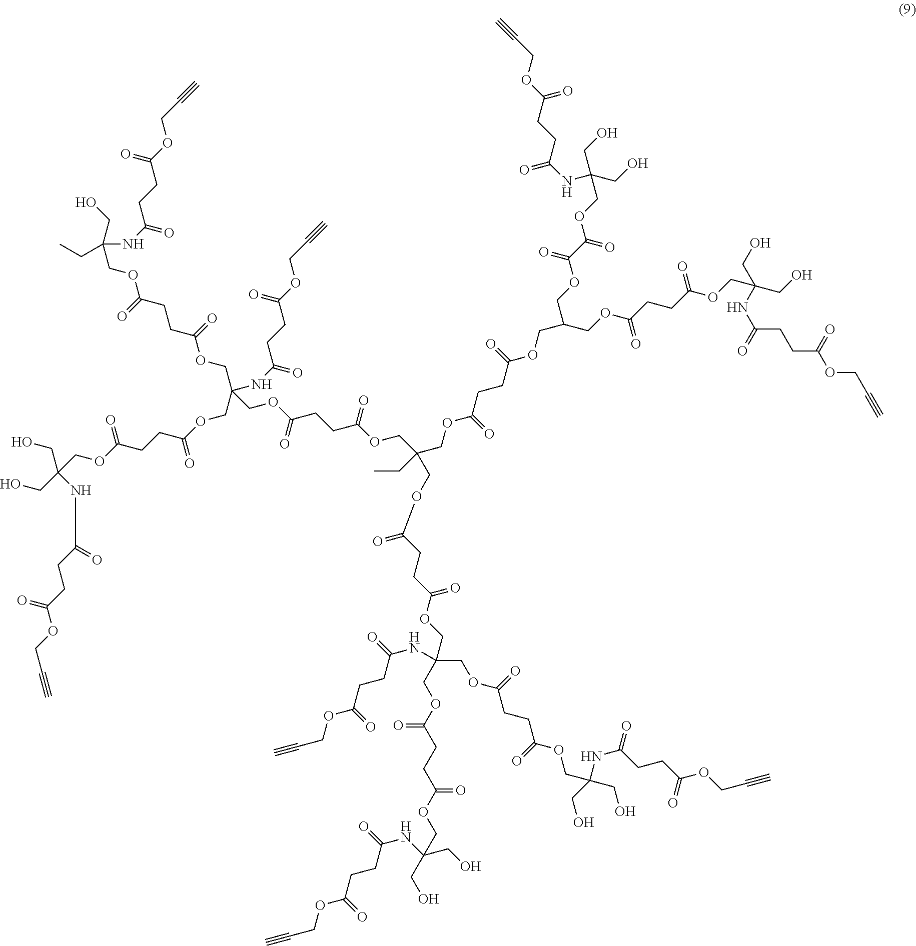 [SMILES: C#CCOC(=O)CCC(=O)NC(CC)(CO)COC(=O)CCC(=O)OCC(COC(=O)CCC(=O)OCC(CC)(COC(=O)CCC(=O)OCC(COC(=O)CCC(=O)OCC(CO)(CO)NC(=O)CCC(=O)OCC#C)COC(=O)C(=O)OCC(CO)(CO)NC(=O)CCC(=O)OCC#C)COC(=O)CCC(=O)OCC(COC(=O)CCC(=O)OCC(CO)(CO)NC(=O)CCC(=O)OCC#C)(COC(=O)CCC(=O)OCC(CO)(CO)NC(=O)CCC(=O)OCC#C)NC(=O)CCC(=O)OCC#C)(COC(=O)CCC(=O)OCC(CO)(CO)NC(=O)CCC(=O)OCC#C)NC(=O)CCC(=O)OCC#C]

### Synthesis of Dendrimer TMP-G3-(Acet)₂₁-(Ac)₁₂ (10)

Compound 5 (5.37 g, 13.5 mmol), 9 (3.00 g, 0.93 mmol), DMAP (0.136 g, 1.11 mmol) and DPTS (0.65 g, 2.22 mmol) were dissolved in anhydrous DCM. DCC (2.75 g, 13.5 mmol) was added to the 0° C. solution. The reaction was left over night and then analyzed using MALDI-TOF to make sure that fully substitution of hydroxyl groups had occurred. The slurry was filtered of, extracted with 10 wt % NaHSO₄ in H₂O and then concentrated. The crude oil was purified by flash chromatography eluting the product in 5/95:MeOH/EtOAc. The product was obtained as a colourless oil after removal of solvent. Yield was 85% (6.15 g).

¹H-NMR (CDCl₃, 400 MHz) δ 0.86 (t, 3H, —CH₃), 1.38 (s, 36H, —CH₃), 1.47 (s, 38H, —CH₂C— and —CH₂C—), 2.47-267 (m, 189H, —CCH, —CH₂CH₂C—), 3.77 (d, 24H, J=12.0 Hz, —CH₂O—), 4.01 (s, 6H, —CH₂C—, core), 4.18 (d, 24H, J=12.0 Hz, —CH₂O—), 4.41 (m, 74H, —CH₂O— and —OCH₂—), 4.66 (m, 42H, —OCH₂CCH—), 6.16 (s, 9H, —CONH—), 6.34 (s, 6H, —CONH—) and 6.41 (s, 3H, —CONH—) ppm.

¹³C-NMR (CDCl₃, MHz) δ 7.33, 22.80, 24.26, 28.78, 28.91, 28.97, 30.92, 30.7, 52.11, 52.83, 57.78, 62.28, 62.57, 63.61, 75.12, 75.21, 77.20, 77.61, 98.67, 171.58, 171.63, 171.72, 171.76, 171.88, 171.99, 172.04, 172.08, 172.34 ppm.

MALDI: Calc. [M_{W}+Na⁺]=7800.79 g/mol, Found [M_{W}+Na⁺]=7803.05 g/mol.

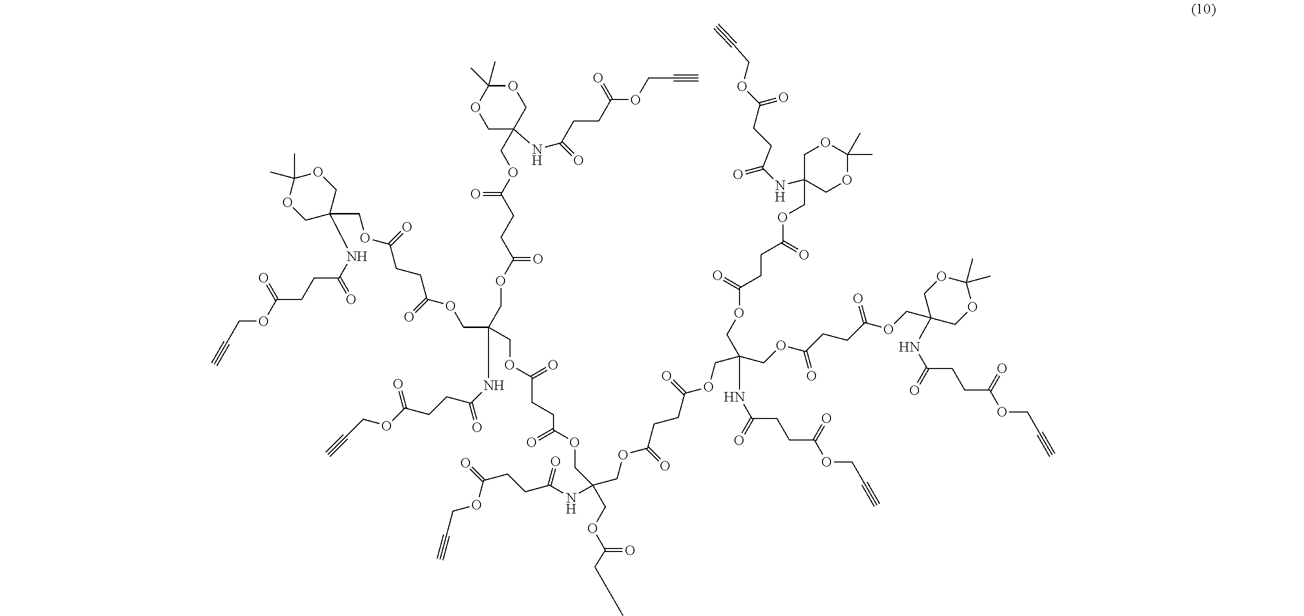 [SMILES: C#CCOC(=O)CCC(=O)NC(COC(=O)CC)(COC(=O)CCC(=O)OCC(COC(=O)CCC(=O)OCC1(NC(=O)CCC(=O)OCC#C)COC(C)(C)OC1)(COC(=O)CCC(=O)OCC1(NC(=O)CCC(=O)OCC#C)COC(C)(C)OC1)NC(=O)CCC(=O)OCC#C)COC(=O)CCC(=O)OCC(COC(=O)CCC(=O)OCC1(NC(=O)CCC(=O)OCC#C)COC(C)(C)OC1)(COC(=O)CCC(=O)OCC1(NC(=O)CCC(=O)OCC#C)COC(C)(C)OC1)NC(=O)CCC(=O)OCC#C]

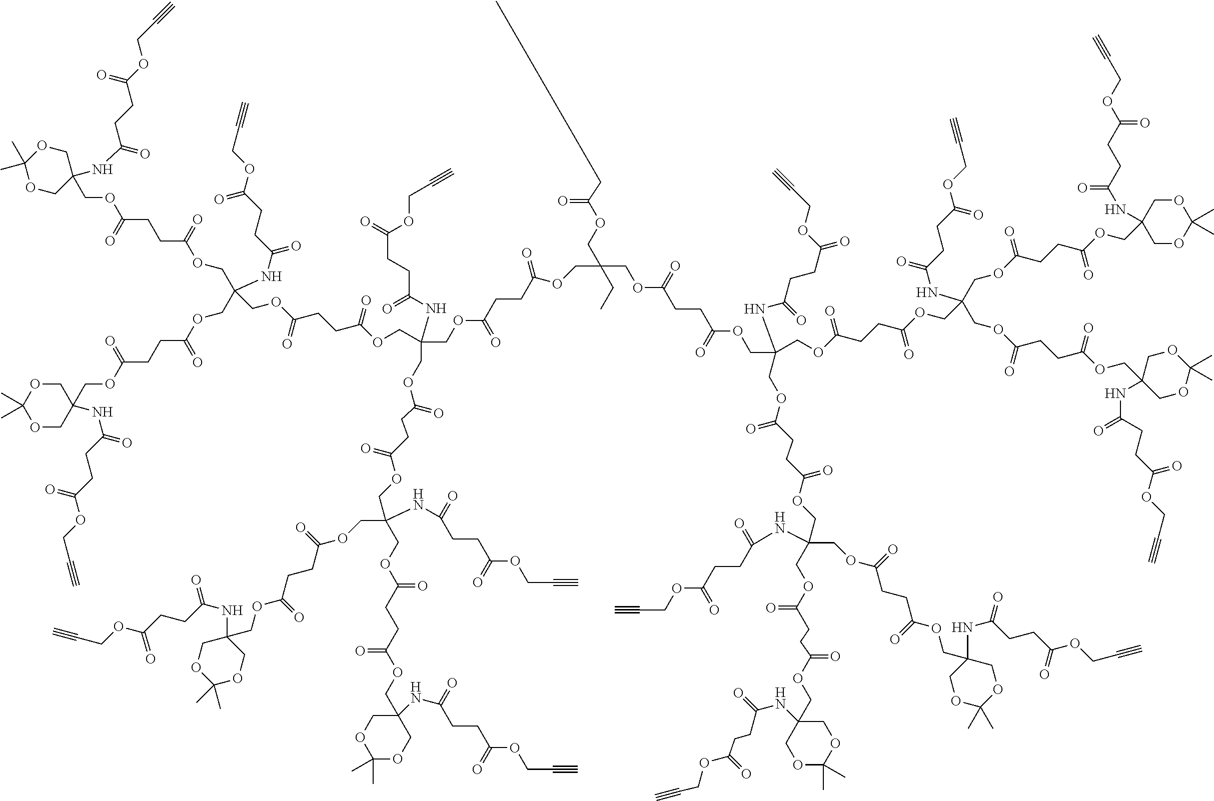

### II. Typical Growth of an Ester Based Acetylene Functional Dendrimer Using TMP Core Unit

### Synthesis of Dendrimer Ar-G1-Acet-Ac (11)

Compound 5 (15.00 g, 37.6 mmol), 1,1,1-tris(4-hydroxyphenyl)ethane (3.20 g, 10.4 mmol), DMAP (0.382 g, 3.13 mmol) and DPTS (1.84 g, 6.27 mmol) were dissolved in anhydrous DCM and cooled down to 0° C. followed by addition of DCC (7.75 g, 37.6 mmol). The reaction was left over night and then analyzed using MALDI-TOF to make sure that fully substitution of hydroxyl groups had occurred. The slurry was filtered off, extracted with NaHSO₄ and then concentrated. The crude oil was purified by flash chromatography eluting the product in 40/60-EtOAc/heptane. The product was obtained as a white solid after removal of solvent. Yield 79% (11.9 g).

¹H-NMR (CDCl₃, 400 MHz) δ 1.38 (s, 9H, —CH₃), 1.48 (s, 9H, —CH₃), 2.14 (s, 3H, Ar—CCH₃), 2.40 (t, 6H, —CH₂—), 2.46 (t, 3H, —CCH), 2.60 (t, 6H, —CH₂—), 2.75 (t, 6H, —CH₂—), 2.91 (t, 6H, —CH₂—), 3.76 (d, 6H, J=12 Hz, —CH₂O—), 4.23 (d, 6H, J=12 Hz, —CH₂O—), 4.52 (s, 6H, —CH₂O—), 4.66 (d, 6H, J=2.4 Hz, —CH₂CCH), 5.92 (s, 3H, —CONH—) and 6.96-7.08 (m, 12H, ArH) ppm.

¹³C-NMR (CDCl₃, MHz) δ 24.79, 24.30, 28.93, 29.28, 31.03, 52.12, 53.11, 62.21 63.74, 75.00, 77.55, 98.71, 120.82, 129.59, 146.13, 148.69, 171.10, 171.65, 171.90, 172.30 ppm.

MALDI: Calc. [M_{W}+Na⁺]=1472.55 g/mol, Found [M_{W}+Na⁺]=1472.62 g/mol.

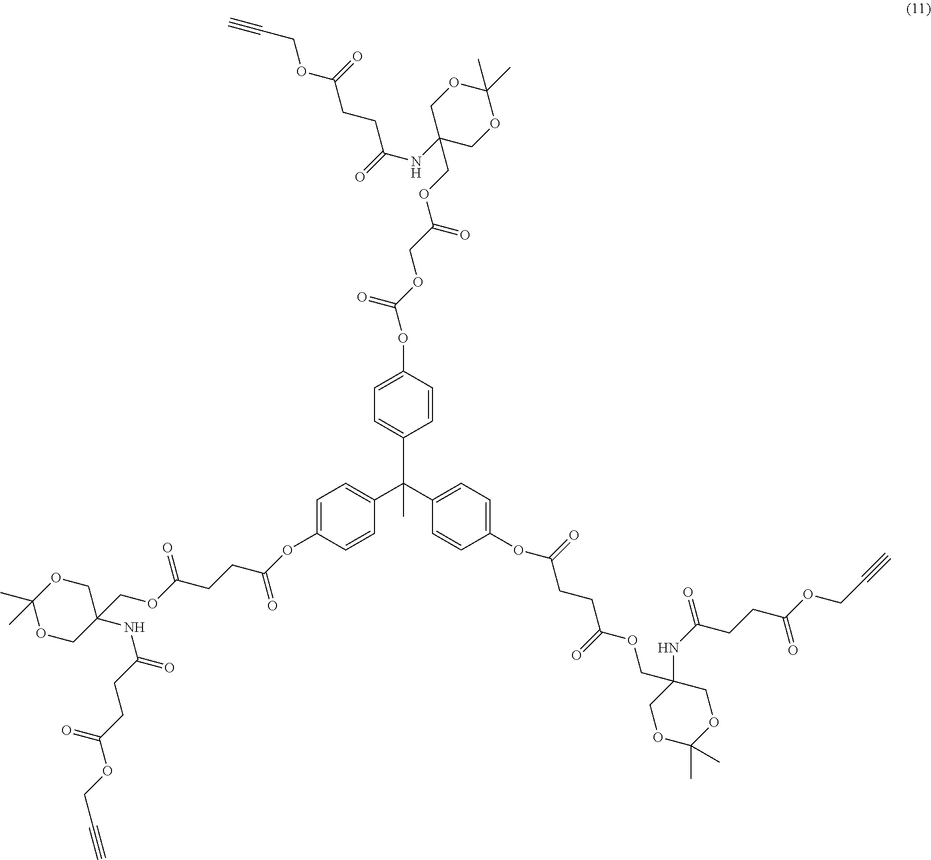 [SMILES: C#CCOC(=O)CCC(=O)NC1(COC(=O)CCC(=O)Oc2ccc(C(C)(c3ccc(OC(=O)CCC(=O)OCC4(NC(=O)CCC(=O)OCC#C)COC(C)(C)OC4)cc3)c3ccc(OC(=O)OCC(=O)OCC4(NC(=O)CCC(=O)OCC#C)COC(C)(C)OC4)cc3)cc2)COC(C)(C)OC1]

### Synthesis of Dendrimer Ar-G1-Acet-OH (12)

Dendrimer 11 (12.00 g, 8.35 mmol) was dissolved in methanol (150 ml) and heated to 45° C. followed by addition of 15 g DOWEX® 50W-X2. The reaction was monitored with TLC and MALDI-TOF. The product was purified by flash chromatography eluting the product in 4/96-methanol/EtOAc. A white powder was obtained after removal of solvent. Yield 90% (10.0 g).

¹H-NMR (DMSO-d₆, 400 MHz) δ 2.14 (s, 3H, Ar—CCH₃), 2.39-2.50 (m, 12H, —CH₂—), 2.67 (t, 6H, —CH₂—), 2.83 (t, 6H, —CH₂—), 3.51 (t, 3H, —CONH—), 3.56 (q, 2H, —CH₂O—), 4.65 (d, 6H, J=2.4 Hz, —CH₂CCH), and 7.04-7.10 (m, 12H, ArH) ppm.

¹³C-NMR (DMSO-d₆, 400 MHz) δ 28.67, 28.77, 30.13, 51.21, 51.59, 59.94, 60.32, 62.28, 77.57, 78.52, 121.14, 129.21, 145.86, 148.52, 170.85, 171.44, 171.59, 171.70 ppm.

MALDI: Calc. [M_{W}+Na⁺]=1352.56 g/mol, Found [M_{W}+Na⁺]=1353.36 g/mol.

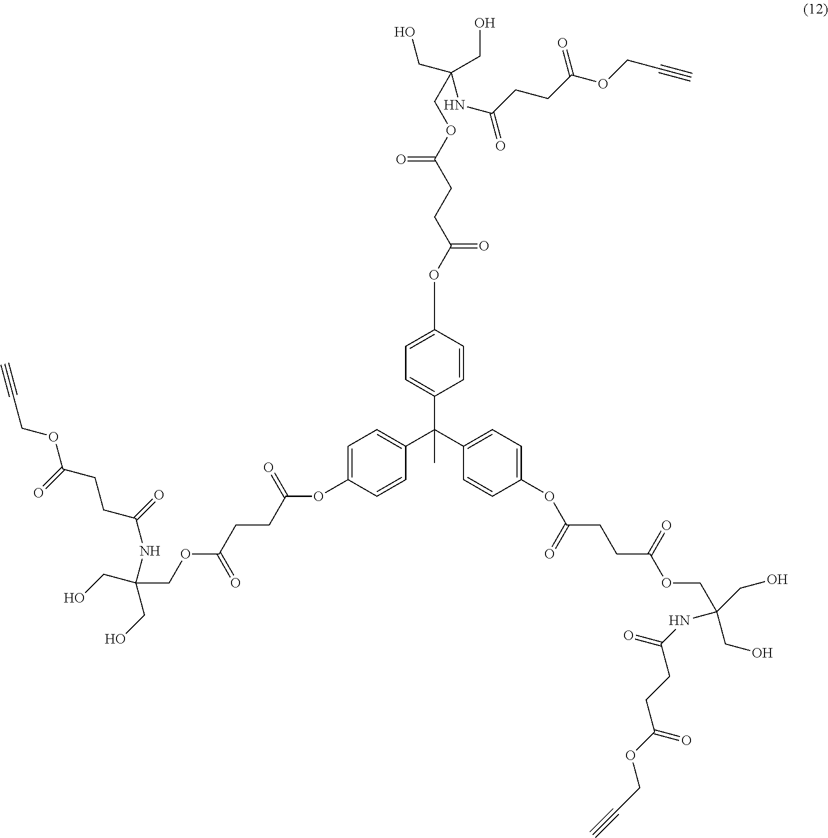 [SMILES: C#CCOC(=O)CCC(=O)NC(CO)(CO)COC(=O)CCC(=O)Oc1ccc(C(C)(c2ccc(OC(=O)CCC(=O)OCC(CO)(CO)NC(=O)CCC(=O)OCC#C)cc2)c2ccc(OC(=O)CCC(=O)OCC(CO)(CO)NC(=O)CCC(=O)OCC#C)cc2)cc1]

### Synthesis of Dendrimer Ar-G2-Acet-Ac (13)

Dendrimer Ar-G1-OH 12 (3.76 g, 2.83 mmol) was mixed with monomer 5 (8.12 g, 20.4 mmol), DMAP (0.207 g, 1.70 mmol), pyridine (2 ml) and DPTS (0.993 g, 3.39 mmol) were dissolved in anhydrous DCM and cooled down to 0° C. followed by addition of DCC (4.20 g, 20.4 mmol). The reaction was left over night and then analyzed using MALDI-TOF to make sure that fully substitution of hydroxyl groups had occurred. The slurry was filtered off, extracted with NaHSO₄ and then concentrated. The crude oil was purified by flash chromatography eluting the product in 60/40-EtOAc/heptane. The product was obtained as a white solid after removal of solvent. Yield was 83% (8.5 g).

¹H-NMR (CDCl₃, 400 MHz) δ 1.38 (s, 9H, —CH₃), 1.48 (s, 9H, —CH₃), 2.14 (s, 3H, Ar—CH₃), 2.40 (t, 6H, —CH₂—), 2.46 (t, 3H, —CCH), 2.60 (t, 6H, —CH₂—), 2.75 (t, 6H, —CH₂—), 2.91 (t, 6H, —CH₂—), 3.76 (d, 6H, J=12 Hz, —CH₂O—), 4.23 (d, 6H, J=12 Hz, —CH₂O—), 4.52 (s, 6H, —CH₂O—), 4.66 (d, 6H, J=2.4 Hz, —CH₂CCH), 5.92 (s, 3H, —CONH—) and 6.96-7.08 (m, 12H, ArH) ppm.

³C-NMR (CDCl₃, MHz) δ 24.79, 24.30, 28.93, 29.28, 31.03, 52.12, 53.11, 62.21 63.74, 75.00, 77.55, 98.71, 120.82, 129.59, 146.13, 148.69, 171.10, 171.65, 171.90, 172.30 ppm.

MALDI: Calc. [M_{W}+Na⁺]=3639.31 g/mol, Found [M_{W}+Na⁺]=3640.44 g/mol.

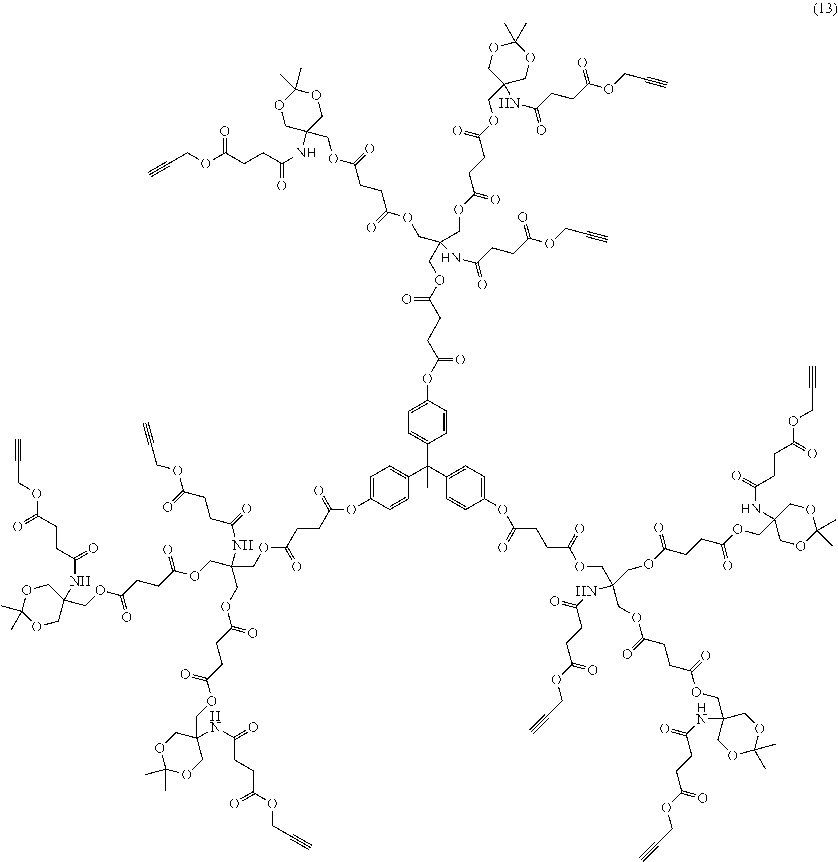

### III. Typical Growth of an Ester Based Acetylene Functional Dendrimer Using Di-TMP Core Unit

### Synthesis of Dendrimer Di-TMP-G1-Acet-Ac (14)

Compound 5 (15.3 g, 38.4 mmol), di-TMP (2.0 g, 8.0 mmol), DMAP (0.39 g, 3.2 mmol) and DPTS (1.87 g, 6.4 mmol) were dissolved in anhydrous DCM and cooled down to 0° C. followed by addition of DCC (7.93 g, 38.4 mmol). The reaction was left over night and then analyzed using MALDI-TOF to make sure that fully substitution of hydroxyl groups had occurred. The slurry was filtered of, extracted with NaHSO₄ and then concentrated. The crude oil was purified by flash chromatography eluting the product in 80/20-EtOAc/heptane. The product was obtained as a colourless oil after removal of solvent. Yield was 90% (12.8 g).

¹H-NMR (CDCl₃, 400 MHz) δ 0.83 (t, J=7.37 hz, 6H), 1.38-1.44 (m, —CCH₃, —CCH₂CH₃, 16H), 1.47 (s, —CCH₃, 12H), 2.46-2.67 (m, CCH₂CH₂—, —CCH₂CH₃, 16H), 3.78 (d, J=11.9, —CCH₂O—, 8H), 3.97 (s, —CCH₂O—, 8H), 4.23 (d, J=11.9, —CCH₂O—, 8H), 4.47 (s, —CCH₂O—, 8H), 4.67 (d, J=2.42 Hz, 8H), 6.09 (s, NH, 4H) ppm.

¹³C-NMR (CDCl₃, MHz) δ 7.38, 22.80, 24.26, 28.88, 28.96, 30.99, 36.87, 41.54, 52.10, 52.93, 62.20, 63.59, 64.50, 70.68, 75.01, 77.54, 98.66, 171.66, 171.95. 171.14 and 172.33 ppm.

MALDI: Calc. [M_{W}+Na⁺]=1797.75 g/mol, Found [M_{W}+Na⁺]=1797.97 g/mol.

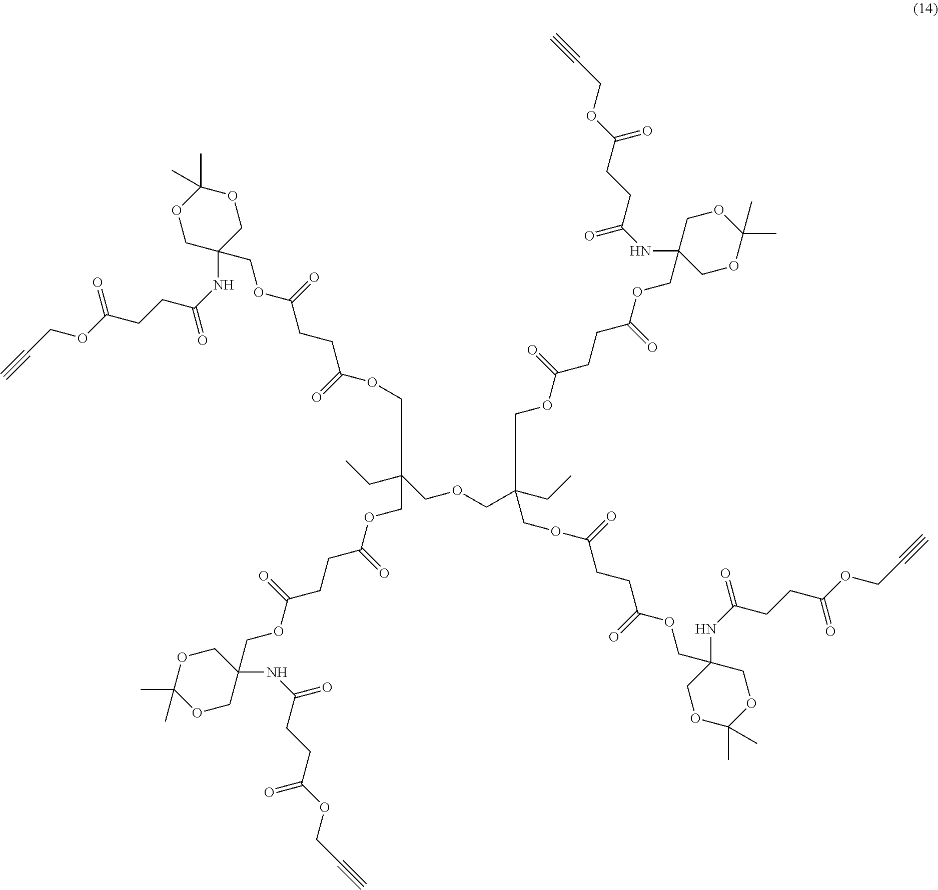 [SMILES: C#CCOC(=O)CCC(=O)NC1(COC(=O)CCC(=O)OCC(CC)(COCC(CC)(COC(=O)CCC(=O)OCC2(NC(=O)CCC(=O)OCC#C)COC(C)(C)OC2)COC(=O)CCC(=O)OCC2(NC(=O)CCC(=O)OCC#C)COC(C)(C)OC2)COC(=O)CCC(=O)OCC2(NC(=O)CCC(=O)OCC#C)COC(C)(C)OC2)COC(C)(C)OC1]

### Synthesis of Dendrimer Di-TMP-G1-Acet-OH (15)

Dendrimer 14 (6.00 g, 3.34 mmol) was dissolved in methanol (150 ml) and heated to 45° C. followed by addition of 10 g DOWEX® 50W-X2. The reaction was monitored with TLC and MALDI-TOF. The product was purified by flash chromatography eluting the product in 1/99-methanol/EtOAc. A colourless oil was obtained after removal of solvent. Yield 92% (4.9 g).

¹H-NMR (DMSO-d₆, 400 MHz) δ 0.88 (t, J=7.37 Hz, 6H), 1.44 (q, —CCH₂CH₃, 2H), 2.43-2.71 (m, CCH₂CH₂—, —CCH₂CH₃, 16H), 3.92 (s, —CCH₂OH, 16H), 4.02 (s, —CCH₂O—, 8H), 4.32 (s, —CCH₂O—, 8H), 4.81 (s, —OCH₂CCH, 8H) ppm.

¹³C-NMR (CDCl₃, MHz) δ 7.42, 28.65, 28.87, 30.77, 36.56, 41.34, 52.19, 52.44, 62.54, 63.23, 64.66, 70.34, 75.66, 77.43, 171.66, 171.95. 171.14, 172.33 ppm.

MALDI: Calc. [M_{W}+Na⁺]=1637.61 g/mol, Found [M_{W}+Na⁺]=1638.02 g/mol.

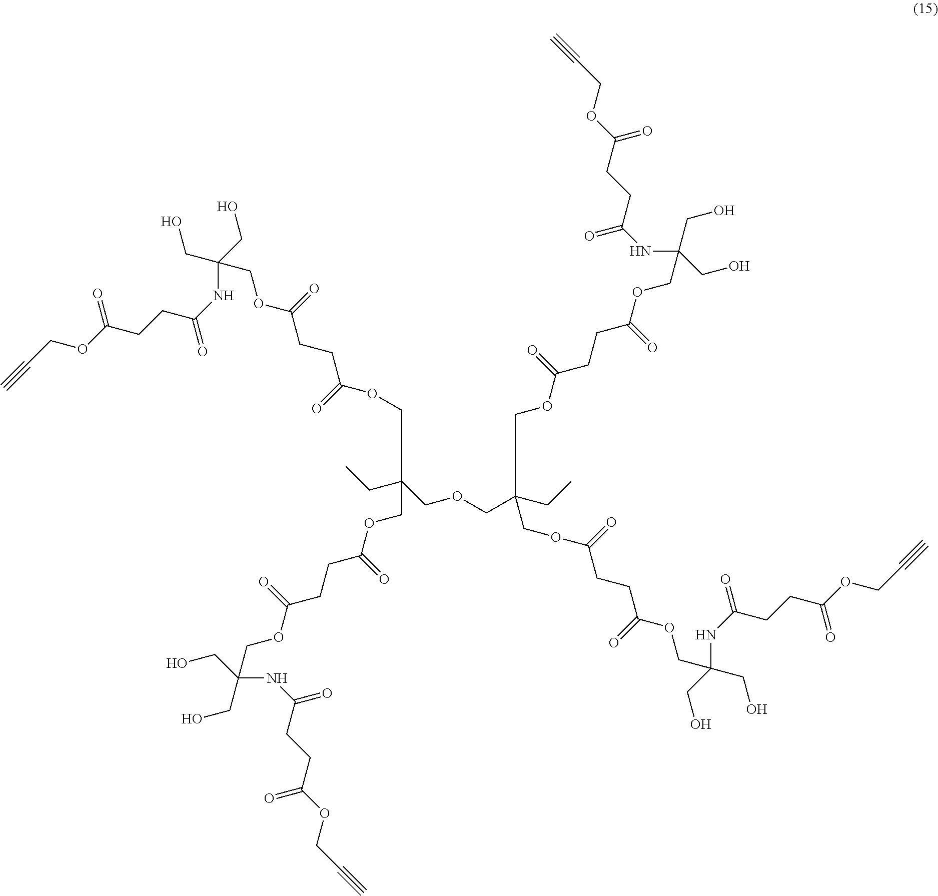 [SMILES: C#CCOC(=O)CCC(=O)NC(CO)(CO)COC(=O)CCC(=O)OCC(CC)(COCC(CC)(COC(=O)CCC(=O)OCC(CO)(CO)NC(=O)CCC(=O)OCC#C)COC(=O)CCC(=O)OCC(CO)(CO)NC(=O)CCC(=O)OCC#C)COC(=O)CCC(=O)OCC(CO)(CO)NC(=O)CCC(=O)OCC#C]

### Synthesis of Ester Based Azide Functional Dendrimers Based on ABₓC_{y} Monomer

### I. Synthesis of the ABₓCₓ Monomer in which

### A is —COOH

### B is —OH

### C is —N₃

x is 2
y is 1

### Synthesis of Acetonide protected 2-(bromomethyl)-2-(hydroxymethyl)propane-1,3-diol (16)

2,2-Dimetoxy propane (31.4 g, 301 mmol) was added to 2-(bromomethyl)-2-(hydroxymethyl)propane-1,3-diol (30 g, 201 mmol) followed by addition of catalytic amount of p-TSA and was left to react over night in 200 ml of acetone. The acid was neutralized with a solution of NH₃/EtOH-mixture followed removal of the solvent. The colorless oil was extracted with DCM/H₂O and dried with MgSO₄. The product was obtained as colorless oil. Yield 88% (63.3 g).

¹H NMR (400 MHz, CDCl₃): δ 1.41 and 1.42 (d, 6H, —CH₃), 3.56 (s, 2H, —CH₂OH), 3.70 and 3.71 (d, 2H, —CH₂Br) and 3.76-3.77 (q, 4H, —CCH₂O—) ppm.

¹³C NMR (CDCl₃) δ 23.2, 23.3, 35.2, 38.4, 62.4, 63.3 and 98.3 ppm.

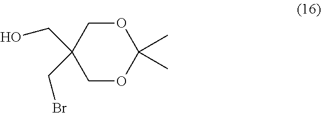

### Synthesis of Acetonide protected 2-(azidomethyl)-2-(hydroxymethyl)propane-1,3-diol (17)

NaN₃ (68.0 g, 1.05 mol) was added to 16 (50.0 g, 209 mmol) and left to react over night in 100 ml of DMSO at 85° C. The solution was left to reach room temperature followed by addition of 50 ml H₂O and extraction 3 times with ether (3*150 ml). The combined organic phases were then extracted with 10 ml of H₂O 2 times. The product was collected after drying with 10 wt % of MgSO₄ in H₂O and rotor evaporation as colorless oil. Yield: 90% (37.8 g).

¹H NMR (400 MHz, CDCl₃): δ 1.41 and 1.42 (d, 6H, —CH₃), 3.56 (s, 2H, —CH₂OH), 3.62 and 3.63 (d, 2H, —CH₂N₃) and 3.69-3.71 (q, 4H, —CCH₂O—) ppm.

¹³C NMR (CDCl₃) δ 23.0, 24.3, 38.9, 52.4, 62.8, 63.0 and 98.5 ppm.

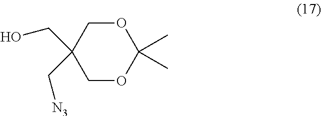

### Synthesis of COOH—N₃-Ac, Acid functionalized acetonide protected 2-(azidomethyl)-2-(hydroxymethyl)propane-1,3-diol (18)

Succinic acid anhydride (30.1 g, 298 mmol) was added to 17 (50 g, 248 mmol) together with DMAP (6.08 g, 59.8 mmol) dissolved in 100 ml of DCM. The reaction was kept over night at room temperature. 100 ml of THF and 20 ml of H₂O was added to the flask to quench the anhydride. The crude solution was extracted 3 times with NaHSO₄ (10 wt %) in water and then dried with MgSO₄. The product was obtained as white powder after removal of solvent. Yield 94% (70.2 g).

¹H NMR (400 MHz, CDCl₃): δ 1.41 (d, 6H, —CH₃), 2.66 (m, 4H, —CH₂CH₂—) 3.50 (s, 2H, —CH₂N₃), 3.69 (q, 4H, —CH₂N₃) and 4.10 (s, 2H, —CCH₂O—) ppm.

¹³C NMR (CDCl₃) δ 22.8, 24.3, 28.7, 28.8, 37.8, 62.7, 63.9, 98.7, 171.7 and 177.6 ppm.

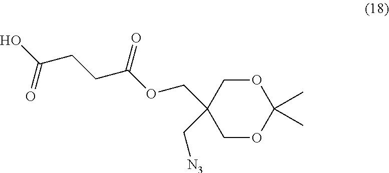 [SMILES: CC1(C)OCC(CN=[N+]=[N-])(COC(=O)CCC(=O)O)CO1]

### II. Typical Growth of an Ester Based Azide Functional Dendrimer Using TMP Core Unit

### Synthesis of TMP-G1-(N₃)₃-(Ac)₃ (19)

TMP (2.00 g, 14.9 mmol) was freeze dried and left to react with 18 (16.17 g, 53.7 mmol) together with DMAP (546 mg, 4.47 mmol), DPTS (2.62 g, 8.94 mmol) and DCC (11.08 g, 53.7 mmol) in DCM. The reaction and purification was performed as the general procedure. The product was eluted in a mixture of 50:50 EtOAc:Hep and obtained as colorless oil. Yield: 90% (13.2 g).

¹H NMR (400 MHz, CDCl₃): δ 0.87 (t, 3H, —CH₃, core), 1.39 (s, 18H, —CH₃), 1.45 (q, 2H, —CH₂—, core), 2.63 (s, 12H, —CH₂CH₂—), 3.50 (s, 6H, —CH₂N₃), 3.69 (q, 12H, —CH₂O—), 4.02 (s, 6H, —OCH₂C—) and 4.07 (s, 6H, —OCH₂C—, core) ppm.

¹³C NMR (CDCl₃) δ 7.3, 22.7, 22.8, 24.3, 28.7, 28.8, 37.7, 40.7, 51.9, 62.7, 63.8, 64.0, 98.5 and 171.7 ppm.

MALDI: Calc. [M_{W}+Na⁺]=1007.01 g/mol, Found [M_{W}+Na⁺]=1006.98 g/mol.

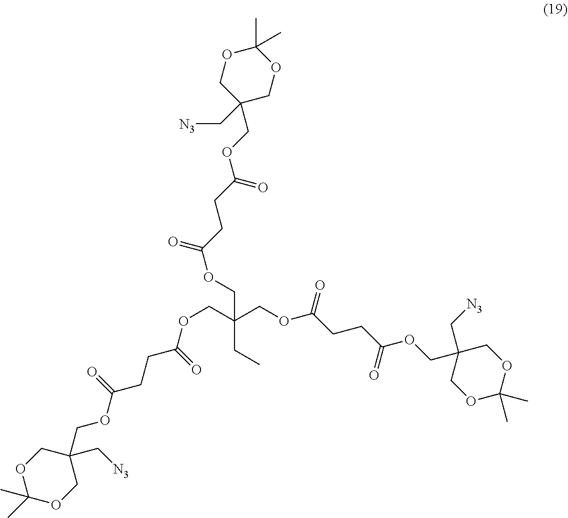

### Synthesis of TMP-G1-(N₃)₃—(OH)₆ (20)

The deprotection of 19 (10 g, 10.2 mmol) was performed as in the general procedure for deprotecting acetonide protected dendrimers. Yield: 98% (8.6 g).

¹H NMR (400 MHz, MeOD-d₄): δ 0.93 (t, 3H, —CH₃, core), 1.53 (q, 2H, —CH₂—, core), 2.68 (s, 12H, —CH₂CH₂—), 3.45 (s, 6H, —CH₂N₃), 3.54 (q, 12H, —CH₂O—), 4.07 (s, 6H, —OCH₂C—) and 4.09 (s, 6H, —OCH₂C—, core) ppm.

¹³C NMR (MeOD-d₄) δ 7.7, 23.9, 29.8, 42.0, 46.0, 52.2, 62.7, 64.5, 65.1, 173.6 and 173.8 ppm.

MALDI: Calc. [M_{W}+Na⁺]=886.82 g/mol, Found [M_{W}+Na⁺]=886.52 g/mol.

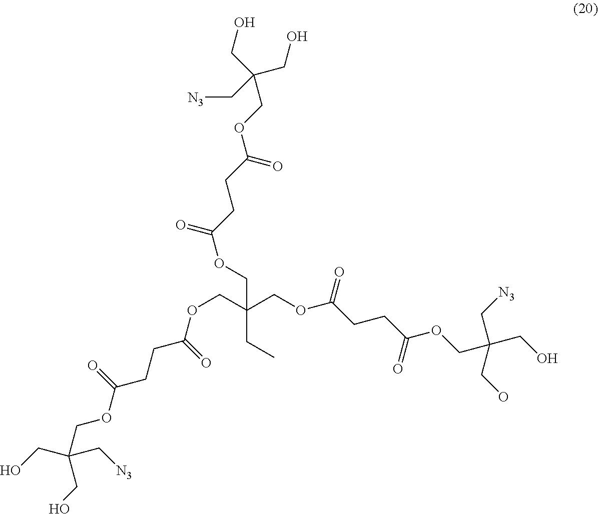 [SMILES: CCC(COC(=O)CCC(=O)OCC(CO)(CO)CN=[N+]=[N-])(COC(=O)CCC(=O)OCC(CO)(CO)CN=[N+]=[N-])COC(=O)CCC(=O)OCC(CO)(CO)CN=[N+]=[N-]]

### Synthesis of TMP-G2-(N₃)₉-(Ac)₆ (21)

Product 20 (6 g, 6.94 mmol) was left to react with 18 (15.1 g, 50.0 mmol) together with DMAP (509 mg, 4.2 mmol), DPTS (2.40 g, 8.2 mmol) and DCC (10.3 g, 50.0 mmol) in DCM. The reaction and purification was performed as the general procedure. The product was eluted in a mixture of 60:40 EtOAc:Hep and obtained as colorless oil. Yield: 85% (15.1 g).

¹H NMR (400 MHz, CDCl₃): δ 0.81 (t, 3H, —CH₃, core), 1.34 (s, 36H, —CH₃), 1.43 (q, 2H, —CH₂—, core), 2.59 (s, 36H, —CH₂CH₂—), 3.41 (s, 6H, —CH₂N₃), 3.45 (s, 12H, —CH₂N₃), 3.64 (q, 24H, —CH₂O—), 3.98 (s, 6H, —OCH₂C—) and 4.03 (m, 30H, —OCH₂C—) ppm.

¹³C NMR (CDCl₃) δ 7.1, 22.4, 22.6, 24.1, 28.6, 28.7, 37.6, 42.4, 50.9, 51.7, 62.4, 62.5, 63.6, 63.8, 98.4, 171.3, 171.4 and 171.5 ppm.

MALDI: Calc. [M_{W}+Na⁺]=2586.51 g/mol, Found [M_{W}+Na⁺]=2586.17 g/mol.

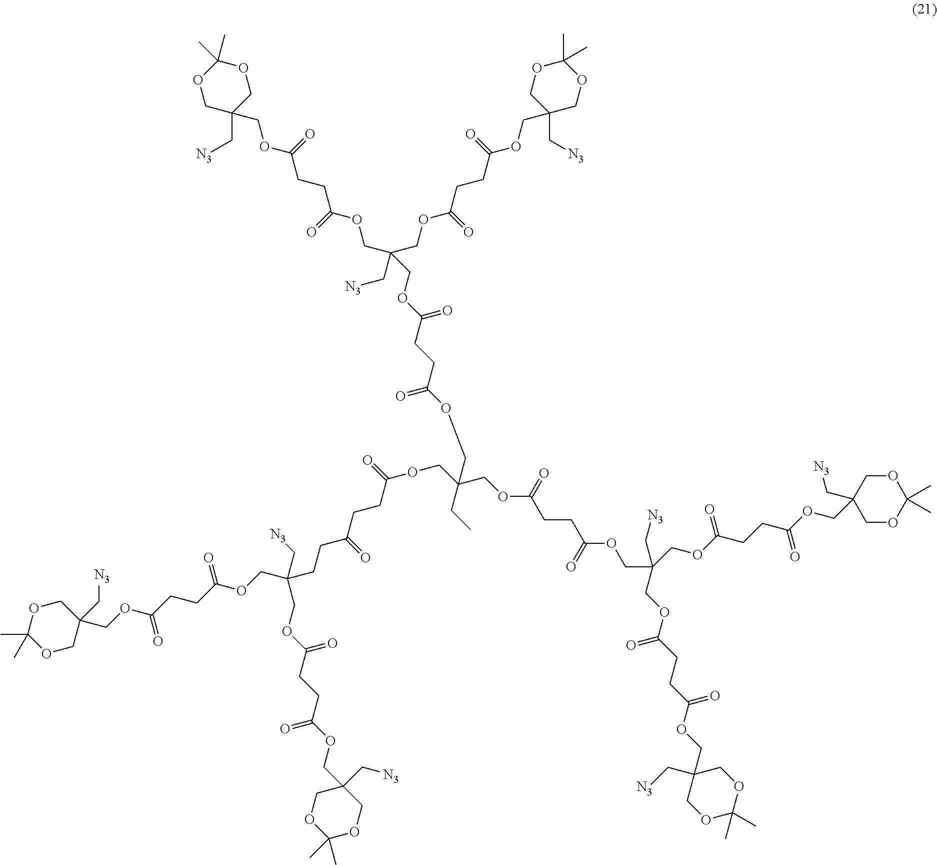

### Synthesis of TMP-G2-(N₃)₉—(OH)₁₂ (22)

The deprotection of 21 (10 g, 3.9 mmol) was performed as in the general procedure for deprotecting acetonide protected dendrimers. Yield: 97% (8.8 g).

¹H NMR (400 MHz, MeOD-d₄): δ 0.95 (t, 3H, —CH₃, core), 1.52 (q, 2H, —CH₂—, core), 2.69 (s, 36H, —CH₂CH₂—), 3.44 (s, 12H, —CH₂N₃), 3.53 (q, 24H, —CH₂O—), 3.56 (s, 6H, —CH₂N₃), 4.07 (s, 12H, —OCH₂C— in G#2), 4.08 (s, 6H, —OCH₂C—, core) and 4.14 (s, 18H, —OCH₂C— in G#1 and —OCH₂C— in G#2) ppm.

¹³C NMR (MeOD-d₄) δ 7.8, 24.1, 29.9, 42.1, 43.9, 46.1, 52.3, 52.4, 61.8, 64.0, 64.6, 65.3, 173.5, 173.6, 173.7 and 173.9 ppm.

MALDI: Calc. [M_{W}+Ag⁺]=2430.12 g/mol, Found [M_{W}+Ag⁺]=2430.90 g/mol.

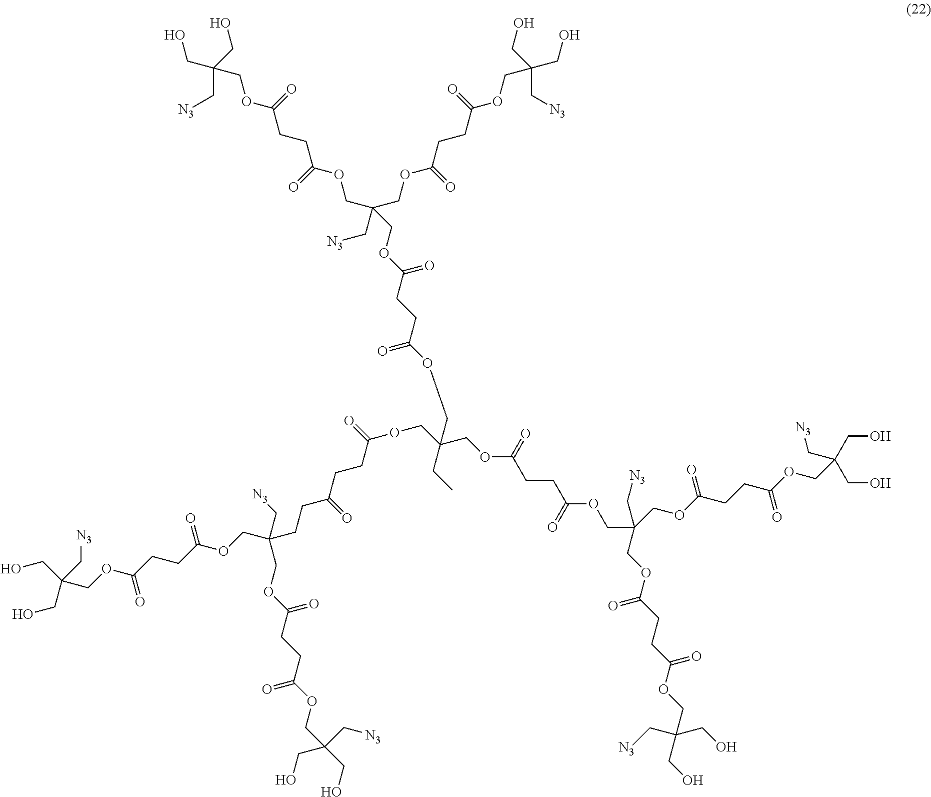 [SMILES: CCC(COC(=O)CCC(=O)CCC(CN=[N+]=[N-])(COC(=O)CCC(=O)OCC(CO)(CO)CN=[N+]=[N-])COC(=O)CCC(=O)OCC(CO)(CO)CN=[N+]=[N-])(COC(=O)CCC(=O)OCC(CN=[N+]=[N-])(COC(=O)CCC(=O)OCC(CO)(CO)CN=[N+]=[N-])COC(=O)CCC(=O)OCC(CO)(CO)CN=[N+]=[N-])COC(=O)CCC(=O)OCC(CN=[N+]=[N-])(COC(=O)CCC(=O)OCC(CO)(CO)CN=[N+]=[N-])COC(=O)CCC(=O)OCC(CO)(CO)CN=[N+]=[N-]]

### Synthesis of TMP-G3-(N₃)₂₁-(Ac)₁₂ (23)

Product 22 (4 g, 1.72 mmol) was left to react with 18 (7.46 g, 24.7 mmol) together with DMAP (252 mg, 2.07 mmol), DPTS (1.21 g, 4.14 mmol) and DCC (5.09 g, 24.7 mmol) in DCM. The reaction and purification was performed as the general procedure. The product was eluted in a mixture of 80:20 EtOAc:Hep and obtained as colorless oil. Yield: 78% (7.7 g).

¹H NMR (400 MHz, CDCl₃): δ 0.85 (t, 3H, —CH₃, core), 1.38 (s, 72H, —CH₃), 1.44 (q, 2H, —CH₂—, core), 2.63 (s, 72H, —CH₂CH₂—), 3.44 (s, 18H, —CH₂N₃ in G#1 and —CH₂N₃ in G#2), 3.48 (s, 24H, —CH₂N₃ in G#3), 3.66 (q, 48H, —CH₂O— in G#3), 4.01 (s, 6H, —OCH₂C—, core) and 4.06-4.07 (m, 78H, —OCH₂C— interior) ppm.

¹³C NMR (CDCl₃) δ 7.2, 22.7, 24.2, 24.8, 28.6, 28.7, 37.7, 40.6, 42.4, 42.5, 49.0, 51.0, 51.8, 62.6, 63.8, 63.9, 98.5, 171.4, 171.5, 171.6 and 171.7 ppm.

MALDI: Calc. [M_{W}+Na⁺]=5745.49 g/mol, Found [M_{W}+Na⁺]=5746.62 g/mol.

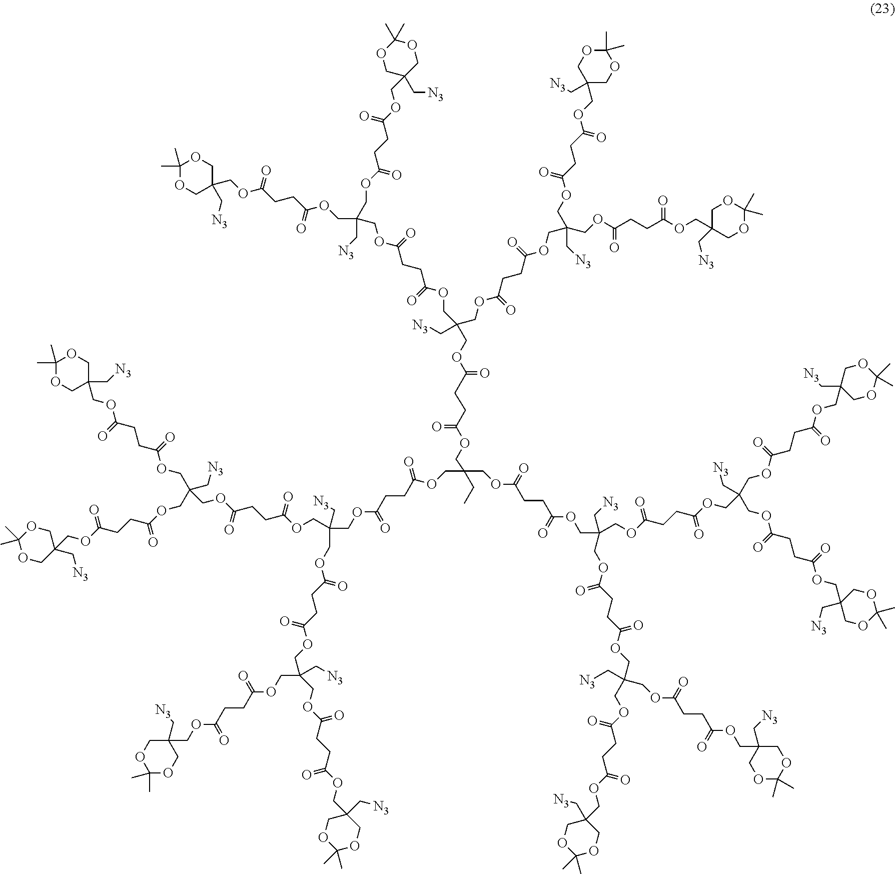

### Example 7

### Post Functionalization Reactions

Multifunctional dendrimers are well suited for one pot post-modifications. These modifications can be prepared in a way which reduces the number of reaction steps for making highly functional materials. This is elucidated by the in-situ model reaction between the 1^{st} generation TMP-G1-Acet₃-OH₆, AB₂C-monomer and benzyl azide. The reaction is carried out in THF using CuSO₄/NaAsc as catalytic system for the reaction and DCC for the esterification reaction. The full substitution of end-groups and intrinsic chemical handles were monitored by MALDI-TOF techniques followed by filtration and purification by preparative chromatography. This one-pot reaction depicts the simplicity of a chemoselective system where the functionalization of the interior and exterior is performed concurrently.

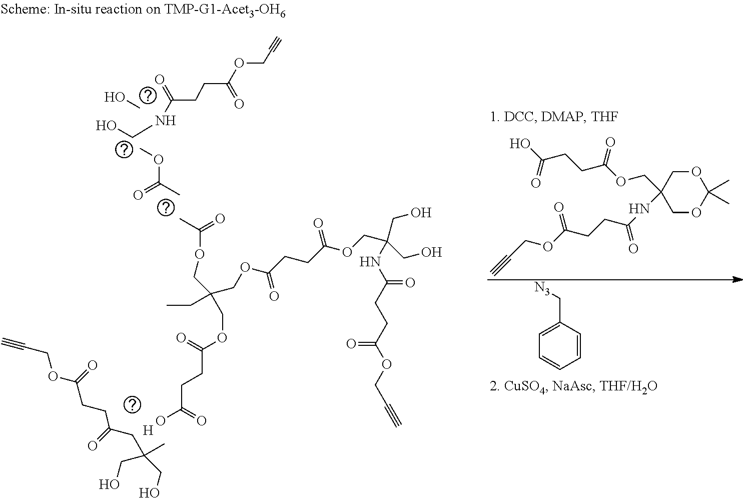

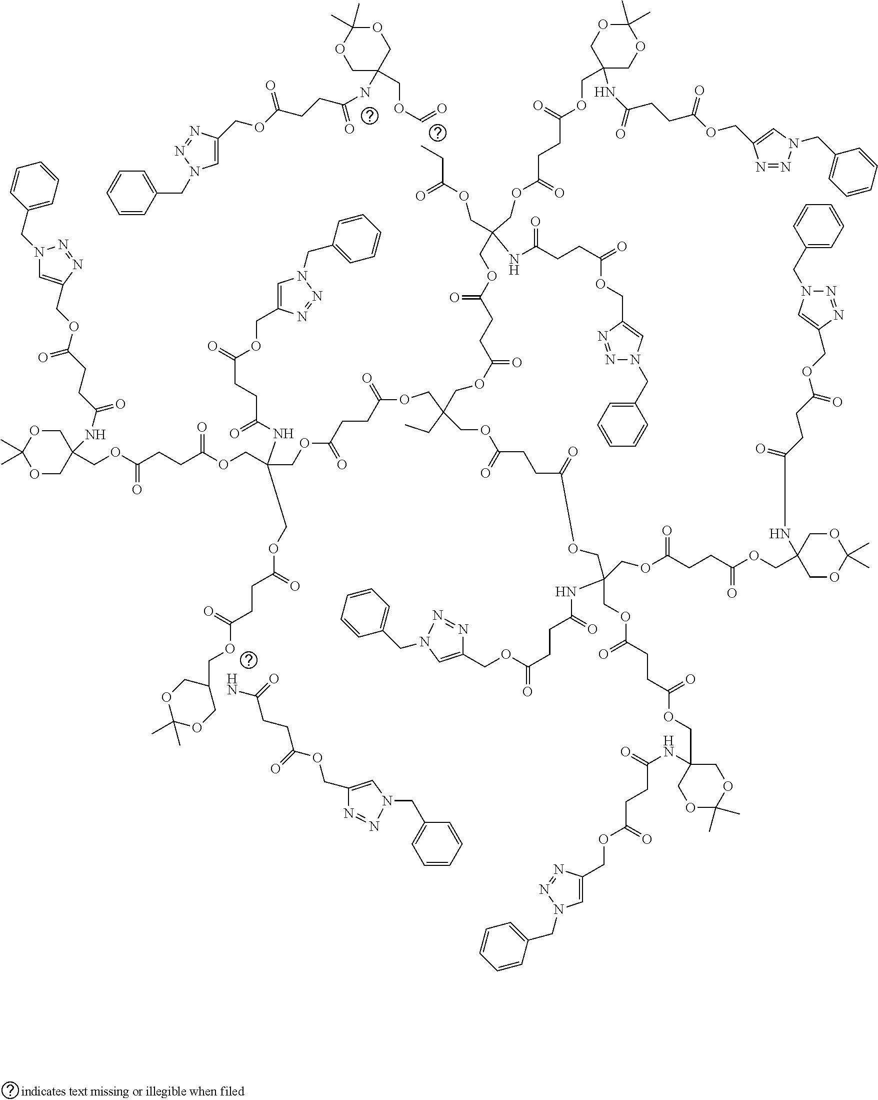

### Example 8

### Post Functionalization Reactions

A second model reaction was performed to further point out the efficiency and facile nature of post-modification of multifunctional dendrimers. The 2^{nd} generation multifunctional dendrimer 9 is treated with the appropriate AB₂C monomer 5 and later with an initiator suited for ATRP (N₃-ATRP). Dendritic growth is obtained using DCC and the post-modification of the interior is performed in THF using CuSO₄/NaAsc as catalytic system. The AB₂C 5 monomer is chosen to depict the facile dendritic growth from the hydroxyl groups at the periphery. Further, instead of using AB₂C monomer to obtain dendritic growth, shorter segments of PEG or other hydrophilic compounds can easily be used to obtain more water soluble dendrimer. The attachment of peripheral groups can be achieved by either DCC coupling or anhydride chemistry. This demonstrates a simple way to add optical, therapeutic, etc. functionality to high generation dendritic structures.

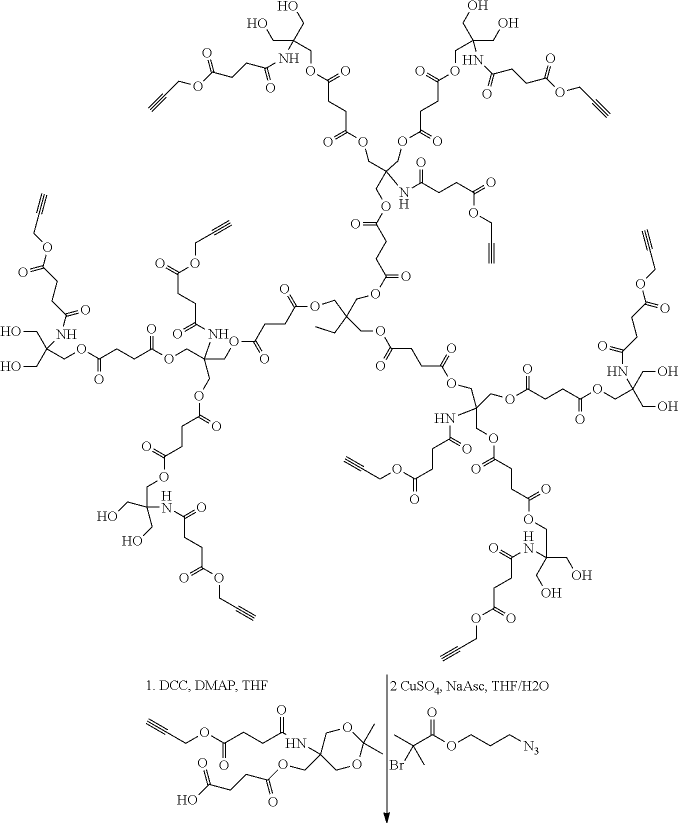

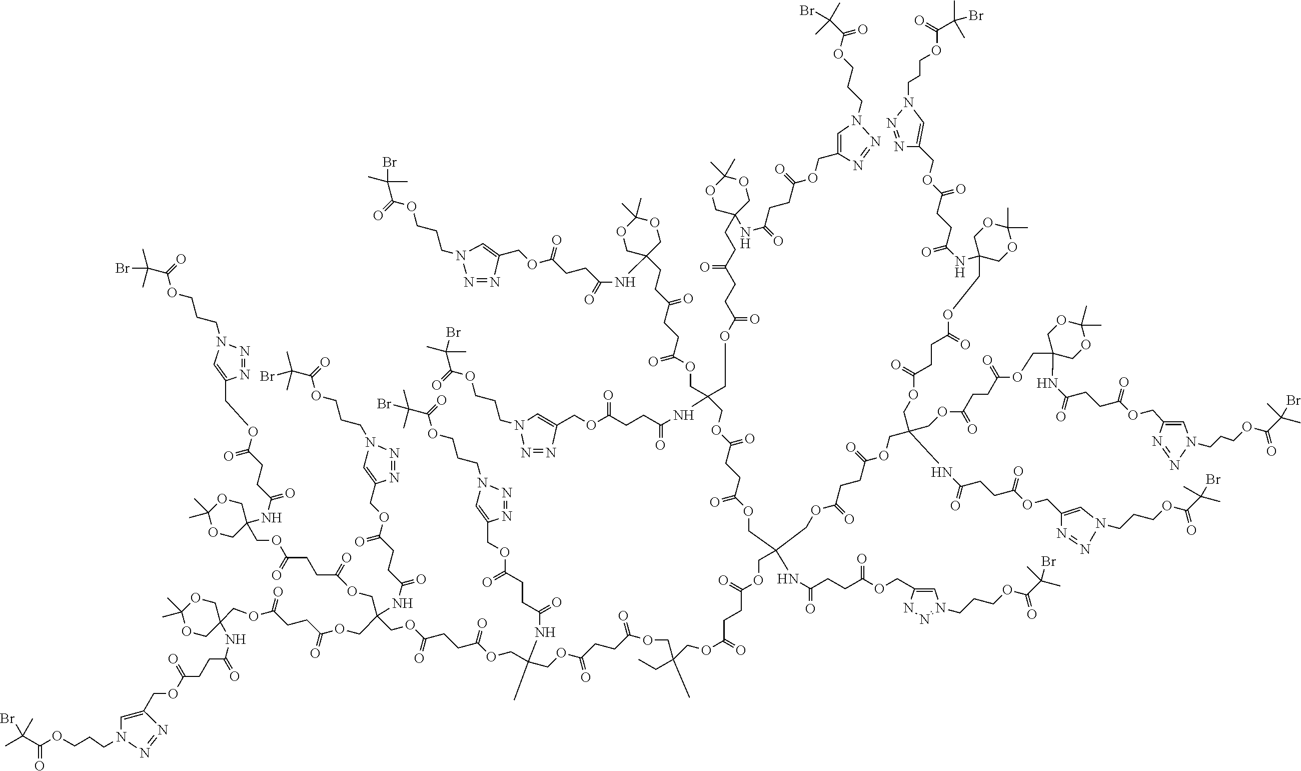

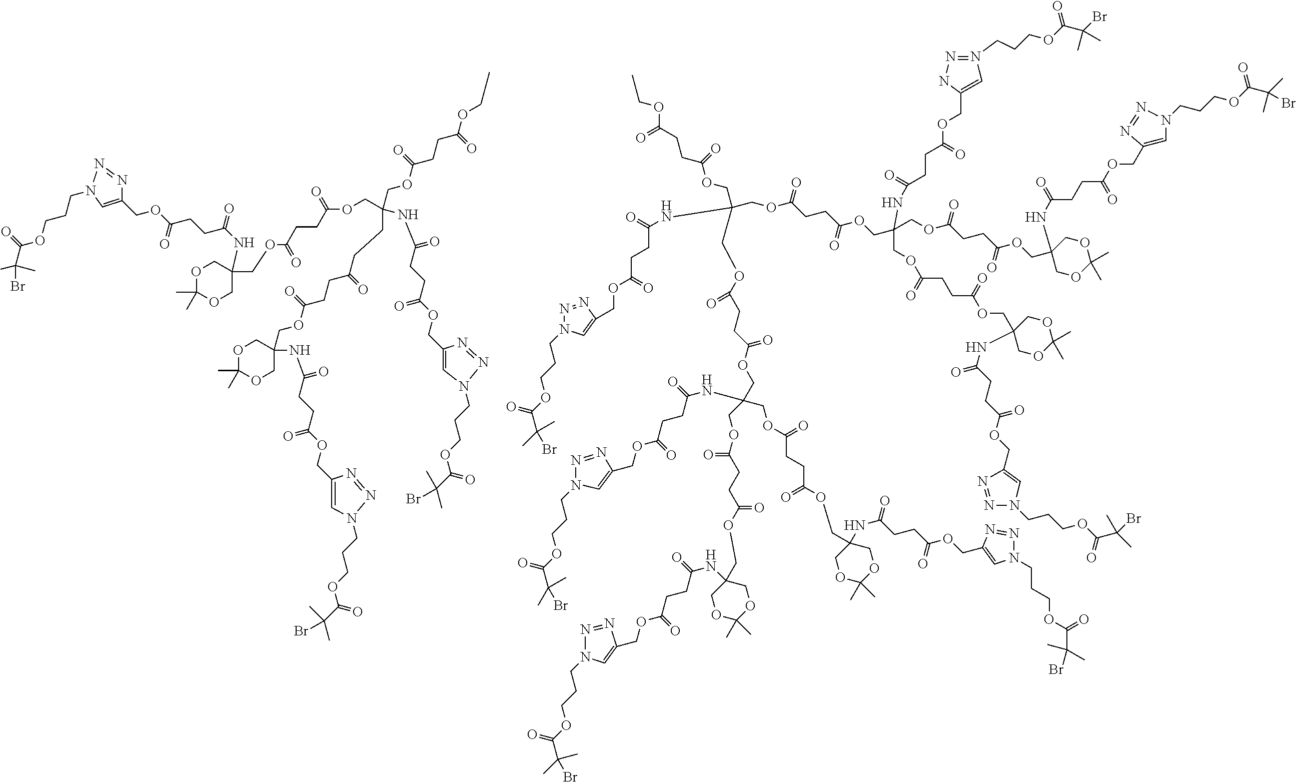

### In-Situ Reaction on TMP-G2-Acet₉-OH₁₂

### Example 9

### Simultaneous Reactions

A model reaction was performed to illustrate the success of simultaneous reactions. 2.9 eqv. of a 3^{rd} generation Bis-MPA dendron with azides at the focal point (1.00 g, 0.958 mmol), 1 eqv. of anthracene anhydride (186 mg, 0.311 mmol) and 1.2 eqv. of an aromatic core consisting of two acetylenes and on hydroxyl group (77.0 mg, 0.358 mmol) were dissolved in CHCl₃. The reaction demands a catalyst in order to proceed and here a Cu(PPh₃)₃Br/DIPEA (0.149 mmol/0.30 mmol) system was used. The completion of the reaction was monitored using ¹H-NMR, ¹³C-NMR and MALDI-TOF. This reaction was performed in a simultaneous manner, where all reactants and reagents were added at once, and resulted in a photo-functional dendrimer. The obtained dendrimer was purified by flash chromatography subsequent two simple extraction procedures, Yield: 82% (253 mg).

¹H NMR (400 MHz, CDCl₃): δ 1.09 (s, 24H, —CH₃), 1.95 (s, 6H, —CH₃), 1.20 (s, 12H, —CH₃), 1.30 (d, 48H, J=24 Hz, —CH₃), 1.32 (m, 8H, —CH₂CH₂—), 1.56 (m, 4H, —CH₂CN—), 1.84 (m, 4H, —OCCH₂C—), 2.61 (s, 4H, —CH₂CH₂—), 3.53 (d, 16H, J=12 Hz, —CH₂O—), 4.03 (t, 4H, —CH₂N—), 4.07 (d, 16H, J=12 Hz, —CH₂O—), 4.17-4.27 (m, 24, —CH₂O—), 4.92 (s, 2H, —OCH₂Ar—), 5.08 (s, 4H, —OCH₂C—), 6.09 (s, 2H, —ArCH₂O—), 6.5 (d, 2H, J=12 Hz ArH), 7.42 (M, 6H, ArH and TriazoleH), 7.99 (d, 2H, J=10 Hz, ArH), (d, 2H, J=10 Hz, ArH) and 8.48 (s, 1H, ArH);

¹³C NMR (CDCl₃) δ 28.2, 29.0, 29.1, 29.6, 30.0, 42.0, 46.5, 46.8, 50.2, 53.4, 59.1, 62.0, 64.8, 65.2, 65.8, 65.9, 66.0, 98.0, 101.4, 106.9, 123.8, 125.0, 126.6, 128.3, 128.5, 129.0, 130.9, 131.2, 131.7, 131.8, 131.9, 132.0, 133.0, 138.2, 159.5, 171.7, 171.8, 172.0, 172.3 and 173.4 ppm.

MALDI: [M_{W}⁺]_{Theoretical}=2737.30 g/mol, [M_{W}⁺]_{Found}=2737.44 g/mol.

### Example 10

### Simultaneous Reactions

A fourth model reaction was performed in order to demonstrate the alternative of dendritic growth along with the functionalization of the focal point of a dendron. Here, azido derivatized coumarine is coupled to the first generation Bis-MPA dendron at the same time as the second layer is added. 1 eqv. of azido coumarine (100 mg, 0.367 mmol), 1.1 eqv. of acetylene-Bis-MPA (69.6 mg, 0.404 mmol) and 2.4 eqv. of acetonide protected Bis-MPA anhydride (363 mg, 1.10 mmol) were dissolved in THF followed by catalysis of the reaction by a Cu(PPh₃)₃Br/DIPEA (0.73 □mol/0.15 mmol) system. The reaction was carried out without the use of DMAP, however the temperature had to be elevated to 50° C. Mass spectrometry revealed full conversion of the simultaneous synthesis of dendrons, yield: 91%.

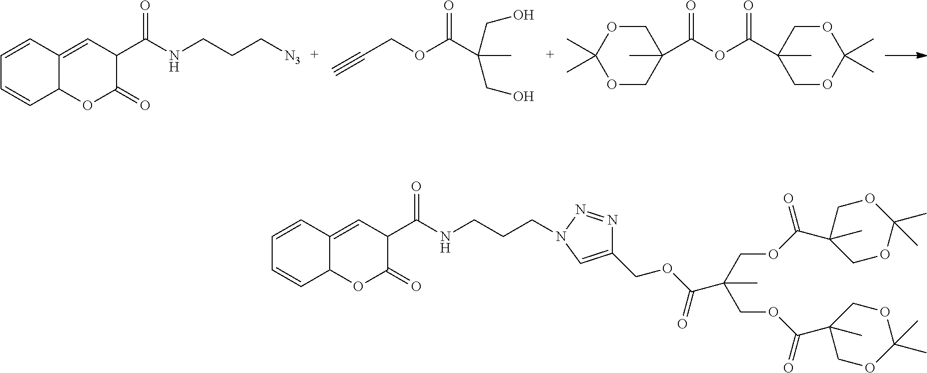

¹H NMR (400 MHz, CDCl₃): δ 1.11 (s, 6H, —CH₃), 1.28 (s, 3H, —CH₃), 1.37 (d, 12H, J=24 Hz, —CH₃), 2.27 (t, 2H, —CNCCH₂CN—), 3.53 (q, 2H, —CNCH₂CCN—), 3.57 (d, 4H, J=12 Hz, —CCH₂O—), 4.95 (d, 4H, J=12 Hz, —CCH₂O—), 4.32 (s, 4H, —CCH₂O—), 4.44 (t, 2H, —CNCCCH₂N—), 5.27 (s, 2H, —CCH₂O—), 7.31-7.67 (m, 4H, ArH), 7.80 (s, 1H, TriazoleH), 8.92 (s, 1H, ArH) and 8.96 (t, 1H, AmideH) ppm;

¹³C NMR (CDCl₃) δ 17.7, 18.5, 22.1, 25.1, 30.3, 36.7, 42.0, 46.8, 47.9, 58.5, 65.2, 65.8, 65.9, 98.0, 116.7, 118.1, 118.6, 124.3, 125.4, 128.4, 128.5, 129.9, 131.9, 132.0, 132.1, 134.3, 142.4, 148.7, 154.4, 161.4, 162.0, 172.5 and 173.5 ppm.

MALDI: [M_{W}⁺]_{Theoretical}=758.34 g/mol, [M_{W}⁺]_{Found}=758.31 g/mol.

### Example 11

### Simultaneous Reactions

Further, the successes in divergent dendritic growth lead to the idea of synthesizing dendrimers in the same divergent manner. Hence, 1 equivalent of a trisphenolic core (200 mg, 0.314 mmol) and 3.3 (1.1/N₃) equivalent of acetylene-Bis-MPA (172 mg, 1.00 mmol) were dissolved in DCM and pyridine followed by addition of a spoon of Cu/C and pyridine (0.5 ml), the reaction was left over night at RT. The Copper/Carbon powder was filtered off where after DMAP (22.9 mg, 0.188 mmol) and 7.9 (1.2/OH) equivalents of the anhydride of the chloro derivative of Bis-MPA were added (530 mg, 2.48 mmol). The reaction was kept over night and then purified using preparative chromatography resulting in the second generation dendrimer, yield: 53%.

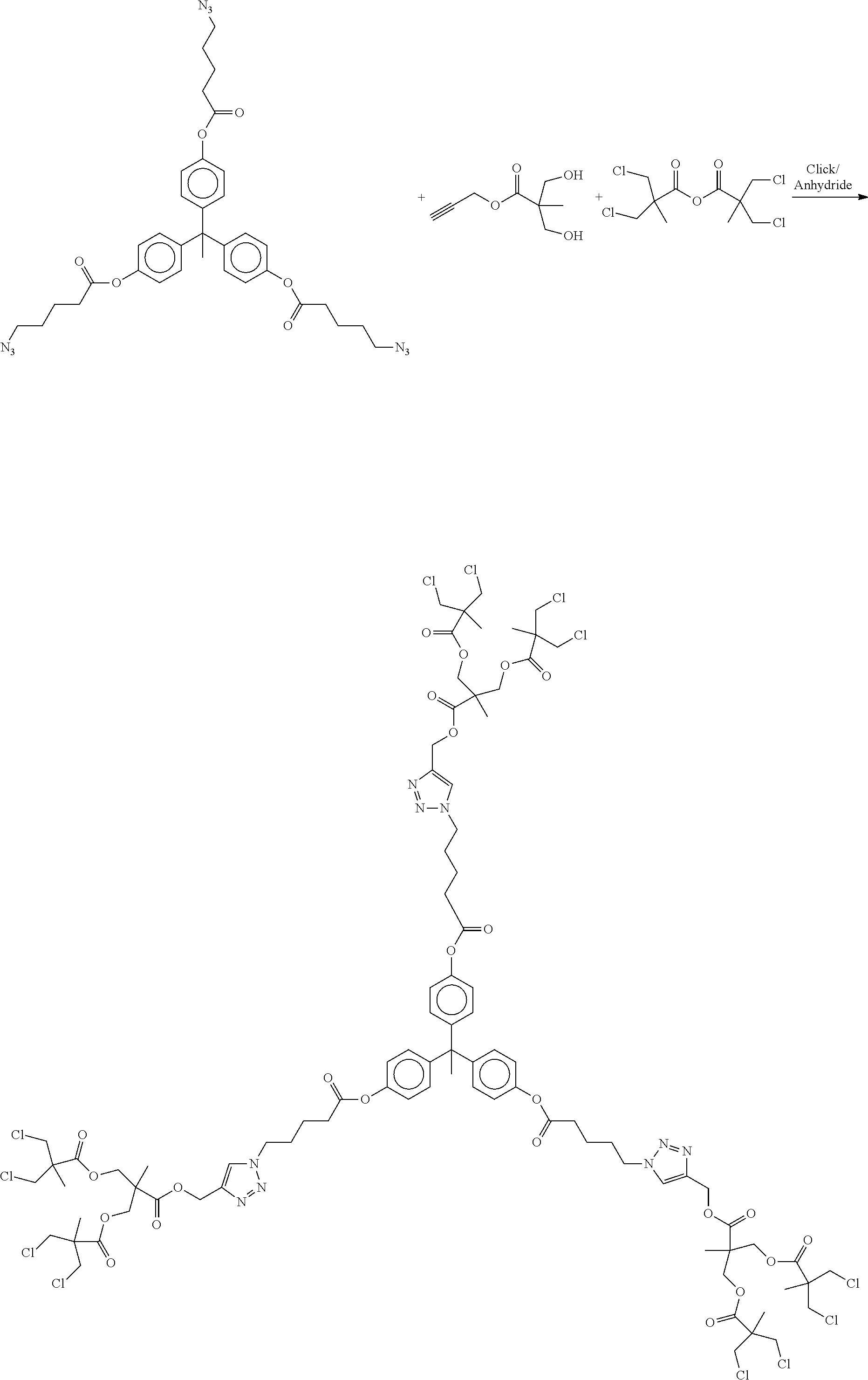

¹H NMR (400 MHz, CDCl₃): δ 1.25 (s, 9H, —CH₃), 1.28 (s, 18H, —CH₃), 2.12 (s, 3H, —CH₃), 2.32 (t, 6H, —CCH₂CN—), 2.60 (t, 6H, —CH₂CCN—), 3.66 (q, 24H, —CH₂O—), 4.28 (q, 12H, —CH₂O—), 4.49 (t, 6H, —CCCH₂N—), 5.25 (s, 6H, —NCH₂O—), 7.02 (q, 12H, ArH), and 7.65 (s, 3H, TriazoleH); ppm;

¹³C NMR (CDCl₃) δ 17.7, 19.8, 25.1, 30.6, 46.2, 46.7, 49.1, 51.5, 58.3, 65.7, 120.7, 124.1, 129.5, 142.0, 146.0, 148.5, 170.7, 171.3 and 172.0 ppm.

MALDI: [M_{W}+Na⁺]_{Theoretical}=2090.35 g/mol, [M_{W}+Na⁺]_{Found}=2091.63 g/mol.

### Example 12

### Hybrid ABₓC_{y} and ABₓ Dendrimers

Hybrid dendrimers were manufactured with a photoactive coumarine core. This was achieved by alternately adding a layer of a traditional ABₓ-monomer and the ABₓC_{y}-monomer. This procedure gives even further control of the amount of functional groups inside the interior. For example, a 3^{rd} generation AB₂C-dendrimer with a trifunctional core would give rise to a dendrimer with 21 interior functionalities and 24 peripheral. By replacing the second layer with AB₂-monomers the amount of functionalities inside the dendrimer may be reduced. The desired number of functional groups in the interior can be achieved simply varying the monomer composition (either ABₓ-monomer or ABₓC_{y}-monomer). This is of great importance in pharmaceutical applications if a high dose of a drug means that it becomes toxic.

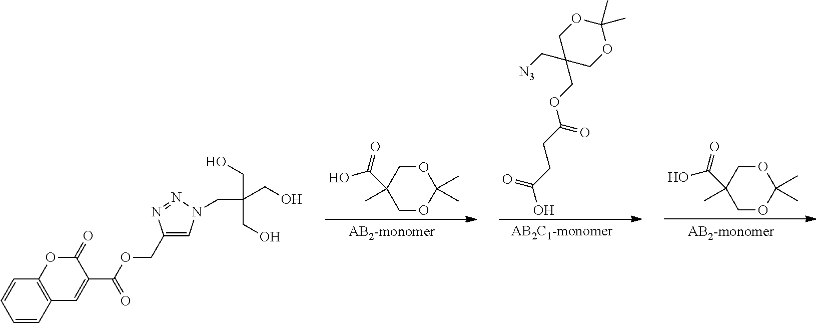 [SMILES: O=C(OCc1cn(CC(CO)(CO)CO)nn1)c1cc2ccccc2oc1=O ; CC1(C)OCC(C)(C(=O)O)CO1 ; CC1(C)OCC(CN=[N+]=[N-])(COC(=O)CCC(=O)O)CO1 ; CC1(C)OCC(C)(C(=O)O)CO1]

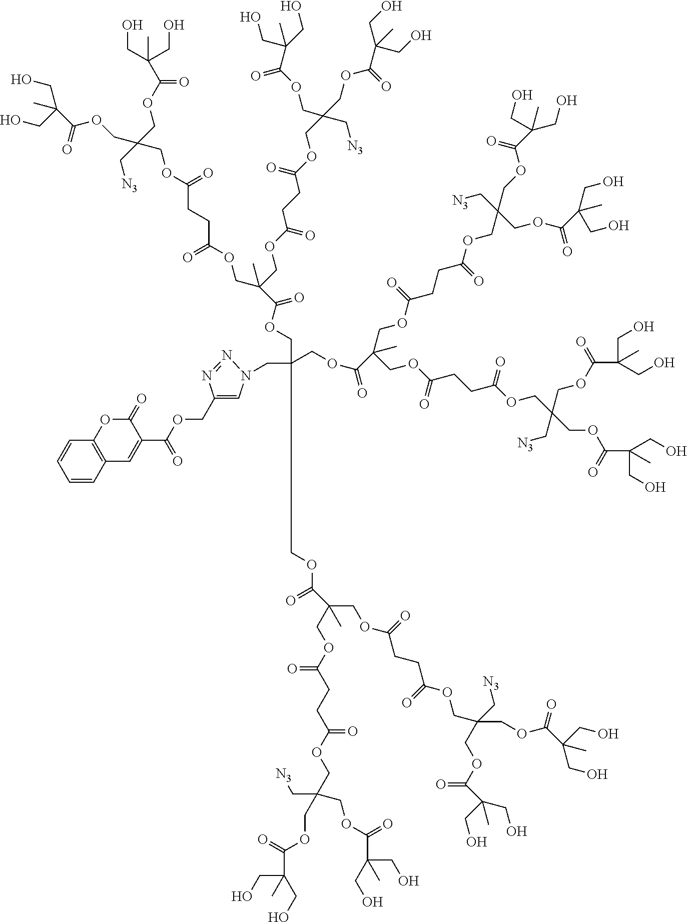 [SMILES: CC(CO)(CO)C(=O)OCC(CN=[N+]=[N-])(COC(=O)CCC(=O)OCC(C)(COC(=O)CCC(=O)OCC(CN=[N+]=[N-])(COC(=O)C(C)(CO)CO)COC(=O)C(C)(CO)CO)C(=O)OCC(COC(=O)C(C)(COC(=O)CCC(=O)OCC(CN=[N+]=[N-])(COC(=O)C(C)(CO)CO)COC(=O)C(C)(CO)CO)COC(=O)CCC(=O)OCC(CN=[N+]=[N-])(COC(=O)C(C)(CO)CO)COC(=O)C(C)(CO)CO)(COC(=O)C(C)(COC(=O)CCC(=O)OCC(CN=[N+]=[N-])(COC(=O)C(C)(CO)CO)COC(=O)C(C)(CO)CO)COC(=O)CCC(=O)OCC(CN=[N+]=[N-])(COC(=O)C(C)(CO)CO)COC(=O)C(C)(CO)CO)Cn1cc(COC(=O)c2cc3ccccc3oc2=O)nn1)COC(=O)C(C)(CO)CO]

### MALDI-TOF Characterization: Tailoring the Molecular Weight

Dendritic structures were synthesized based of different monomer composition and cores to exemplify how the molecular weight can be tailored. MALDI-TOF technique was used to verify the controllability of the molecular weight of the components used. Further, by varying the core functionality to a higher number, the dendritic structure possess higher functional group number.

### Example 13

### Dendritic Nanoparticle Through Intramolecular Collapse Using Azide Crosslinking

Dendrimers equipped with azide interior was exposed to a Fusion UV source. The dendritic structure collapses to a more constrained nano-structure and therefore depicts the possibility of using these multifunctional dendritic structures as entrapping carriers for low molecular weight drugs. Two primary azides will form a nitrene with N₂ as leaving group. This intra-molecular reaction is favoured, in contrast to the inter-molecular, if the reaction is performed under dilute conditions, nanosized spherical objects can be obtained. The crosslinking reaction that occurs inside the dendrimers will shrink the dendrimers to different sizes depending on the intensity of the light and the time for which the dendrimers are being exposed. However, to much intensity and long irradiation time will lead to breaking of the ester backbone and deprotection of the acetonide end-groups. A concentration of 0.5 mg/ml of TMP-G2-(N₃)-Ac₆ in THF (HPLC quality with 2% toluene as internal standard) was put in a quartz cuvette and sealed and was then exposed to UV-light (0.537 J/cm²) under 4 scans. The cuvette was opened after each scan and an aliquot was collected and injected into the GPC. The formation of nitrenes? and decrease in molecular weight corresponding to the loss of N₂-gas was monitored with MALDI-TOF. The decrease in molecular weight and hydrodynamic volume was determined using GPC indicating that smaller molecules are formed. However, even though the lowest concentration possible for detection by GPC was used, a small fraction of inter-molecular cross-linking was observed, 3% after the first scan and up to 7% after 4 scans. These fractions were calculated by integrating the different GPC-traces. The authors believe that this unwanted cross-linking can be avoided if more dilute solutions are used.

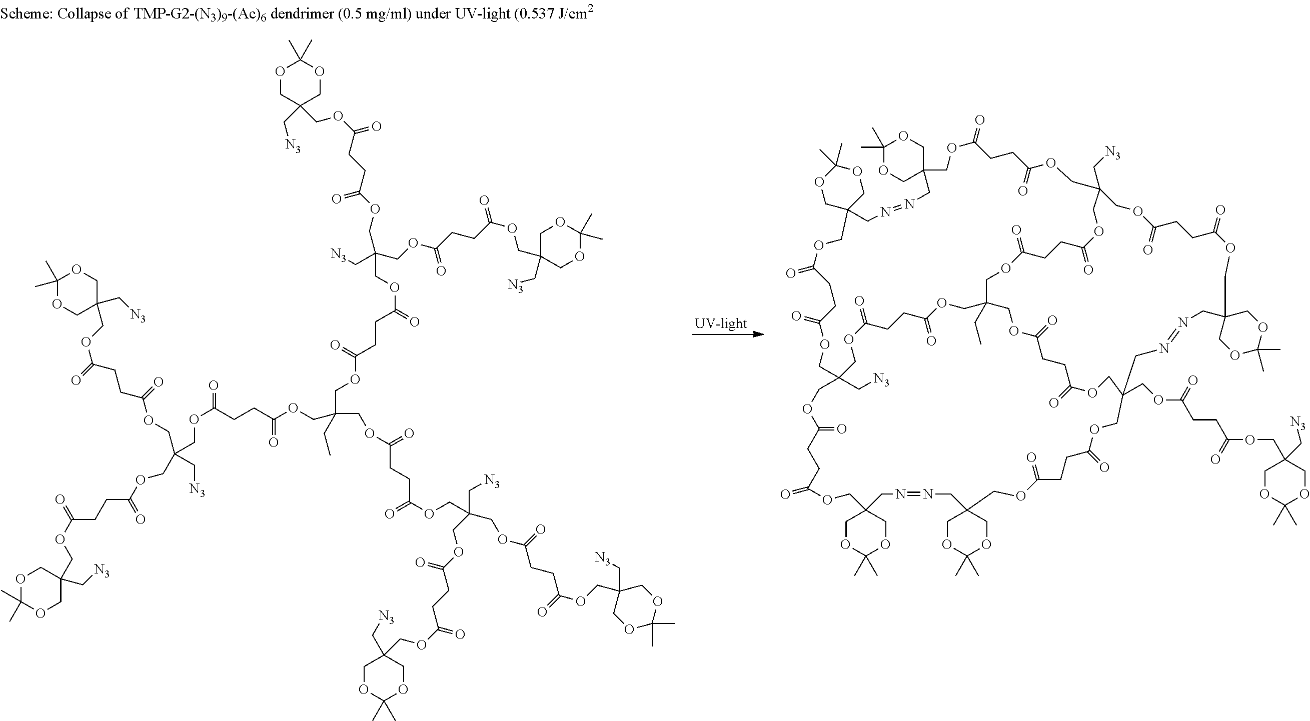

### Example 14

### Dendritic Hydrogels from Multifunctional Dendrimers

These new types of dendrimers form stable hydrogels when crosslinked together with an azido derivatized PEG. The hydrogel will expand and adsorb differently as a result of the crosslinking density and PEG length. We illustrate the formation of hydrogels from these multifunctional dendrimers by reacting a 2^{nd} generation dendrimer with a bifunctional azide PEG. The reaction was catalyzed by a CuSO4/NaAsc system in water. A slightly yellow colored hydrogel could be removed from the mold after 24 hours. It was put in a EDTA/Water solution to extract the Cu out of the hydrogel. The hydrogel was collected as a transparent swollen film. It increased its length by 83%, its weight by almost 2600% water when compared to its dry state and have a water content of 96%.

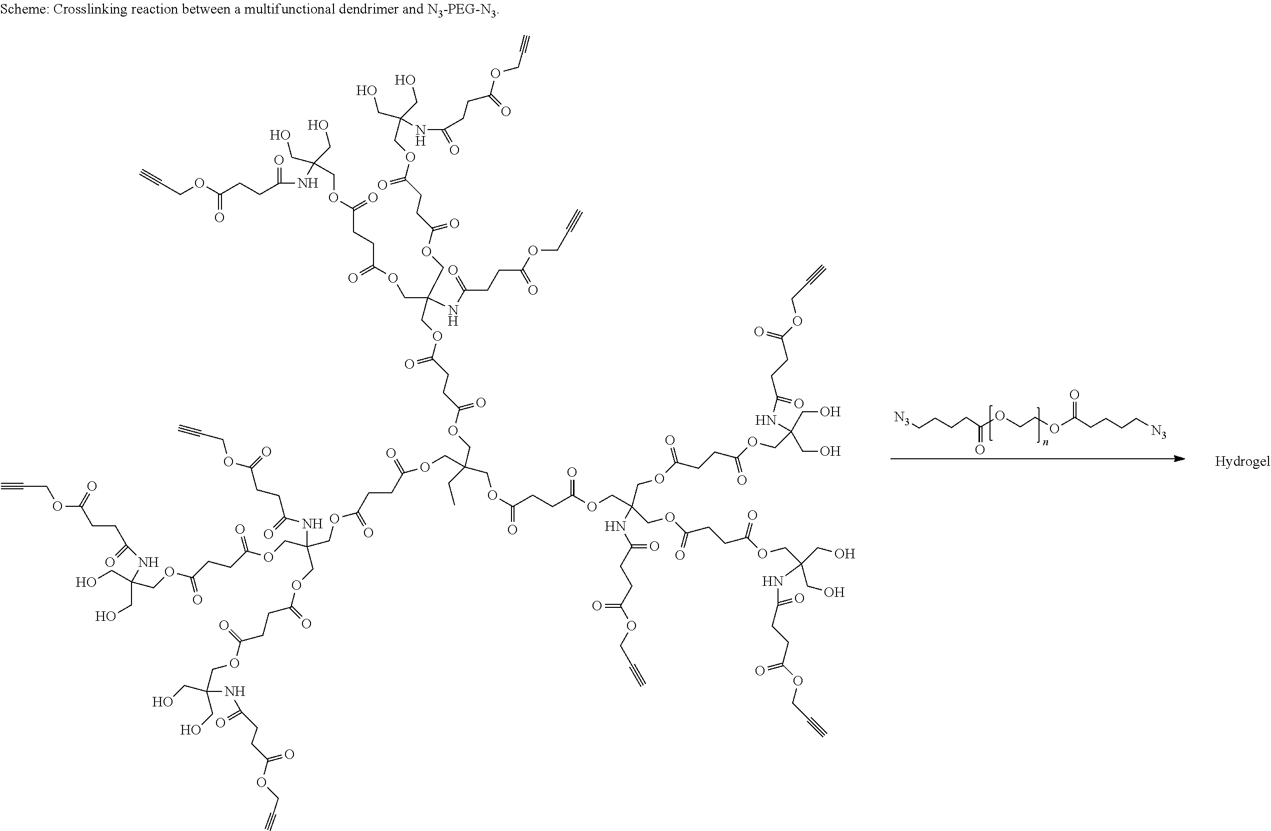

1. A method of manufacturing a dendritic structure comprising the steps:
a) reacting at least two monomers of the type ABₓC_{y} with a core molecule, and
b) reacting the result from step a) with monomers of the type ABₓC_{y} to obtain a dendritic structure,
wherein x is 2, 3, or 4,
wherein y is 1, 2, or 3,
wherein C comprises an entity selected from the group consisting of azides and alkynes, and
wherein every repeating unit is bound to at least one other unit with at least one bond selected from the group consisting of an ester, an amide, a thioether, an ether, a urethane, an amine, and an imine.

2. The method according to claim 1, wherein the step b) is repeated.

3. The method according to claim 1 further comprising the step of adding functional groups to the dendritic structure wherein a functional group is added by reaction with a group C in the dendritic structure.

4. The method according to claim 3 wherein at least two different functional groups are added in one step by reaction with a group C and a group B respectively.

5. The method according to claim 1, wherein A comprises COOH and B comprises OH.

6. The method according to claim 1, wherein A comprises COOH and B comprises NH2.

7. The method according to claim 1, wherein A comprises NCO and B comprises OH.

8. The method according to claim 1, wherein A comprises vinylic and B comprises SH.

9. The method according to claim 1, wherein A comprises N-hydroxysuccinimide (NHS) ester and B comprises NH₂.

10. The method according to claim 1, wherein A comprises a halogen and B comprises OH.

11. The method according to claim 1, wherein B comprises COOH and A comprises OH.

12. The method according to claim 1, wherein B comprises COOH and A comprises NH2.

13. The method according to claim 1, wherein B comprises NCO and A comprises OH.

14. The method according to claim 1, wherein B comprises vinylic and A comprises SH.

15. The method according to claim 1, wherein B comprises N-hydroxysuccinimide (NHS) ester and A comprises NH₂.

16. The method according to claim 1, wherein B comprises a halogen and A comprises OH.

17. The method according to claim 1, wherein x is 2 or 3.

18. The method according to claim 1, wherein x is 2.

19. The method according to claim 1, wherein y is 1 or 2.

20. The method according to claim 1, wherein y is 1.

21. The method according to claim 1, wherein the repeating units are bonded with divergent growth approach.

22. The method according to claim 1, wherein the repeating units are bonded with convergent growth approach.

23. The method according to claim 1, wherein the dendritic structure is selected from the group consisting of a dendrimer, a dendron, a hyperbranched polymer and a dendronized polymer.

24. The method according to claim 1, wherein the dendritic structure is a dendritic structure of at least generation 1.

25. The method according to claim 1, wherein the dendritic structure is a dendritic structure of at least generation 3.

26. The method according to claim 1, further comprising addition of at least one functional group, wherein said at least one functional group is attached by reaction with an azide or alkyne in the dendritic structure.

27. The method according to claim 1, further comprising manufacture of a particle, said method further comprising a crosslinking reaction of dendritic structures.

28. The method according to claim 1, further comprising manufacture of a hydrogel, said method further comprising a crosslinking reaction of dendritic structures.
